(19) United States
(12) Patent Application Publication
(10) Pub. No.: US 2015/0203599 A1
(43) Pub. Date: Jul. 23, 2015

(54) POLYSACCHARIDE SYNTHASES (X)

(71) Applicant: ADELAIDE RESEARCH & INNOVATION PTY LTD, Adelaide (AU)

(72) Inventors: Geoffrey Bruce Fincher, Hazelwood Park (AU); Rachel Anita Burton, Panorama (AU)

(73) Assignee: ADELAIDE RESEARCH & INNOVATION PTY LTD, Adelaide (AU)

(21) Appl. No.: 14/419,364

(22) PCT Filed: Aug. 2, 2013
(86) PCT No.: PCT/AU2013/000854

§ 371 Date: Feb. 3, 2015

(30) Foreign Application Priority Data
Aug. 3, 2012 (AU) 2012903344

(51) Int. Cl. C08B 37/00; C12N 15/82; C12N 9/10; C12P 19/04

(52) U.S. Cl. CPC C08B 37/0057; C08B 37/0003; C12P 19/04; C12N 15/8246; C12N 9/1077; C12Y 204/02024

(57) ABSTRACT

The present disclosure relates generally to polysaccharide synthases. The present disclosure reveals that a subset of the CesA gene family encode XynS xylan synthases. As a result of the identification of XynS nucleic acids, and corresponding amino acid sequences that encode XynS xylan synthases, the present invention provides, inter alia, methods and compositions for modulating the level and/or activity of xylan synthase in a cell and/or modulating the level of xylan produced by a cell.

### CROSS-REFERENCE TO RELATED APPLICATIONS

This application is a 371 of international patent application number PCT/AU2013/000854 entitled “POLYSACCHARIDE SYNTHASES (X)”, filed Aug. 2, 2013, and claims the benefit of Australian patent application number 2012903344 entitled “POLYSACCHARIDE SYNTHASES (X)”, filed Aug. 3, 2012, the contents of each of which are hereby incorporated by reference in their entirety.

### FIELD

The present disclosure relates generally to polysaccharide synthases. More particularly, the present invention relates to xylan synthases.

### BACKGROUND

Heteroxylans are widely distributed as cell wall components in the plant kingdom and are particularly abundant in cell walls of the economically important grass family.

The heteroxylans of the grasses consist of a (1,4)-linked backbone of β-D-xylopyranosyl (Xylp) residues, to which is appended single α-L-arabinofuranosyl (Araf), single α-D-glucuronopyranosyl (GlcpA) residues, or the 4-O-methyl ethers of GlcpA residues. In some cases oligosaccharide substituents such as β-D-Xylp-(1,2)-L-Araf-(1- and β-D-Galp(1,4)-β-D-Xylp-(1,2)-L-Araf-(1- are also detected in heteroxylans. The Araf residues are usually linked to the C(O)3 position of the Xylp residues, but on occasions are found on C(O)2 or on both of these carbon atoms. The GlcpA residues are usually linked to the C(O)2 atom of the Xylp residues. The structural features of selected plant heteroxylans are summarised in FIG. 1.

The number and distribution of substituents along the (1,4)-β-D-xylan backbone vary between and within species, and are major determinants of the physicochemical properties of the heteroxylans. In the heteroxylans from the starchy endosperm of grasses, the major substituents are Araf residues; GlcpA residues are less common. If the (1,4)-β-D-xylan backbone is heavily substituted with Araf residues, the polysaccharide will be more soluble, because the Araf residues sterically inhibit intermolecular alignment of individual molecules and prevent aggregation and precipitation. A proportion of the Araf residues of arabinoxylans from the grasses are substituted at C(O)5 with hydroxycinnamic acids; ferulic acid is the common hydroxycinnamic acid, but p-coumaric acid is also found (see FIG. 1B). Ferulic acid residues account for up to 1.8% (w/w) of wheat aleurone walls and 0.04% of barley starchy endosperm walls. Oxidative coupling of feruloyl residues on adjacent arabinoxylan chains is believed to allow covalent cross-linking of polysaccharide chains in the wall. The heteroxylan from Arabidopsis contains the reducing terminal oligosaccharide 4-β-D-Xylp-(1,4)-β-D-Xylp-(1,3)-α-L-Rhap-(1,2)-α-D-GalpA-(1,4)-D-Xylp. So far there is no evidence that such an oligosaccharide is present in arabinoxylans from the grasses.

Heteroxylans are also present in walls of dicots and monocots other than the Poaceae, where they have structural features similar to those described above but, in many cases, fewer arabinosyl substituents, relatively more glucuronyl residues and few if any feruloyl residues. There are also heteroxylans in these other plants that have unusual monosaccharide and oligosaccharide substituents. For example, the heteroxylan from the mucilage of Plantago ovata is heavily substituted with single xylopyranosyl residues linked to carbon atom 2 of the (1,4)-β-D-xylan backbone and with α-L-Araf-(1,3)-β-D-Xylp-(1,3)-α-Araf-(1 trisaccharides, linked to carbon atom 3 of the (1,4)-β-D-xylan backbone.

Through intense recent efforts, many of the genes that encode glycosyl transferases involved in the addition of substituents to the (1,4)-β-D-xylan backbone have been identified, but there is still a high degree of uncertainty as to the identity of genes that mediate in the synthesis of the (1,4)-β-D-xylan backbone itself. Much of the initial work on the identification of genes involved in heteroxylan synthesis was focused on analyses of Arabidopsis mutant lines and transcript profiling. However, these experiments were plagued with interpretative difficulties imposed by the large gene families, compensation and pleiotropic effects in transgenic lines during proof-of-function tests, and the difficulties associated with developing reliable biochemical assays of expressed enzymes.

In light of the above, identification of the genes involved in the synthesis of the backbone (1,4)-β-D-xylan of plant cell wall heteroxylans would be desirable. Such identification would, among other things, allow the development of cells, including plant cells, and organisms which exhibit modulated levels of heteroxylans.

### DESCRIPTION

The present invention is predicated, in part, on the identification of biosynthetic enzymes, and their encoding genes, that catalyse the synthesis of xylan.

Nucleotide and amino acid sequences are referred to herein by a sequence identifier number (SEQ ID NO:). A summary of the sequence identifiers is provided in Table 1. A sequence listing is provided at the end of the specification.

TABLE 1
Summary of Sequence Identifiers

| Sequence Identifier | Description |
|---|---|
| SEQ ID NO: 1 | PoC321 XynS amino acid sequence |
| SEQ ID NO: 2 | PoC321 XynS nucleotide sequence |
| SEQ ID NO: 3 | PoC217 XynS amino acid sequence |
| SEQ ID NO: 4 | PoC217 XynS nucleotide sequence |
| SEQ ID NO: 5 | PoC420 XynS amino acid sequence |
| SEQ ID NO: 6 | PoC420 XynS nucleotide sequence |

“Xylan” as referred to herein should be understood to encompass polysaccharides which comprise at least (1,4)-linked β-D-xylopyranosyl (Xylp) residues. In some embodiments, the (1,4)-linked β-D-xylopyranosyl residues may form a polysaccharide backbone to which other residues may optionally be appended.

In some embodiments, xylans may incorporate α-L-arabinofuranosyl (Araf), α-D-glucuronopyranosyl (GlcpA) residues, or the 4-O-methyl ethers of GlcpA residues appended to the (1,4)-linked β-D-xylopyranosyl backbone. In some embodiments, other oligosaccharide substituents such as β-D-Xylp-(1,2)-L-Araf-(1-, α-L-Araf-(1,3)-β-D-Xylp-(1,3)-α-Araf-(1, (1,2)-β-D-Xylp and β-D-Galp(1,4)-β-D-Xylp-(1,2)-L-Araf-(1- may also be incorporated into xylans. Xylans comprising residues appended to a (1,4)-linked β-D-xylopyranosyl backbone may also be described as heteroxylans or arabinoxylans or glucuronoxylans or glucuronoarabinoxylans or pentosans (depending on the nature of the backbone substituents) and such compounds should be considered within the scope of the term “xylan” as used herein. Non-limiting examples of xylans contemplated by the present invention are illustrated in FIG. 1.

The biosynthetic enzymes that catalyse the synthesis of xylan are referred to herein as “XynS xylan synthases”. In some embodiments, a “XynS xylan synthase” refers to any protein which at least catalyses the polymerisation of (1,4)-linked β-D-xylopyranosyl residues and/or oligosaccharides.

Herein it is disclosed that particular members of the CesA gene family encode XynS xylan synthases.

The CesA group represents one Glade of the much larger cellulose synthase-like (Csl) gene family (see FIG. 5), which encodes GT2 group enzymes. To date, the CesA gene family has been considered to contain only genes that participate in cellulose synthesis.

However, in accordance with the present invention, it has been revealed that a subset of the CesA gene family encode XynS xylan synthases. Members of the subset of CesA genes that encode XynS xylan synthases are referred to herein as XynS nucleic acids, nucleic acid sequences, nucleotide sequences or genes.

As a result of the identification of XynS nucleic acids, and corresponding amino acid sequences that encode XynS xylan synthases, the present invention provides, inter alia, methods and compositions for modulating the level and/or activity of xylan synthase in a cell and/or modulating the level of xylan produced by a cell.

Therefore, in a first aspect, the present invention provides a method for modulating the level of xylan produced by a cell, the method comprising modulating the level and/or activity of a XynS xylan synthase in the cell.

By “modulating” with regard to the level of xylan is intended decreasing or increasing the level of xylan in and/or produced by the cell.

By “decreasing” is intended, for example, a 1%, 5%, 10%, 20%, 30%, 40%, 50%, 60%, 70%, 80%, 90%, 91%, 92%, 93%, 94%, 95%, 96%, 97%, 98%, 99%, 100% reduction in the level of xylan in or produced by the cell relative to a wild type of the cell.

By “increasing” is intended, for example, a 1%, 5%, 10%, 20%, 30%, 40%, 50%, 60%, 70%, 80%, 90%, 100%, 2-fold, 5-fold, 10-fold, 20 fold, 50-fold, 100-fold increase in the level of xylan in or produced by the cell relative to a wild type of the cell.

“Modulating” also includes introducing xylan production into a cell which does not normally produce xylan, or the substantially complete inhibition of xylan production in a cell that normally produces xylan.

The “cell” may be any suitable eukaryotic or prokaryotic cell. As such, a cell as referred to herein may be a eukaryotic cell including a fungal cell such as a yeast cell or mycelial fungus cell; an animal cell such as a mammalian cell or an insect cell; or a plant cell. Alternatively, the cell may also be a prokaryotic cell such as a bacterial cell including an E. coli cell, or an archaea cell.

In some embodiments, the cell is a plant cell. A “plant cell” as referred to herein encompasses any cell from any organism of the kingdom plantae. This includes, for example, green algae (eg. divisions Chlorophyta and Charophyta), bryophytes (eg. divisions Marchantiophyta, Anthocerophyta and Bryophyta), pteridophytes (eg. divisions Lycopodiophyta and Pteridophyta) and seed plants (eg. divisions Cycadophyta, Gingkophyta, Pinophyta, Gnetophyta and Magnoliophyta).

In some embodiments, the plant cell is a vascular plant cell, including a monocotyledonous or dicotyledonous angiosperm plant cell, or a gymnosperm plant cell. In some embodiments the plant is a monocotyledonous plant cell. In some embodiments, the plant is a member of the order Poales. In some embodiments, the monocotyledonous plant cell is a cereal crop plant cell.

As used herein, the term “cereal crop plant” includes members of the Poales (grass family) that produce edible grain for human or animal food. Examples of Poales cereal crop plants which in no way limit the present invention include wheat, rice, maize, millet, sorghum, rye, triticale, oats, barley, tef, wild rice, spelt and the like. However, the term cereal crop plant should also be understood to include a number of non-Poales species that also produce edible grain and are known as the pseudocereals, such as amaranth, buckwheat and quinoa.

In some embodiments, the cereal crop plant is a barley, wheat or rice plant.

As referred to herein, “barley” includes several members of the genus Hordeum. In some embodiments, the term “barley” includes cultivated barley species such as Hordeum distichum, Hordeum tetrastichum and Hordeum vulgare. In some embodiments, barley may also refer to wild barley (eg. Hordeum spontaneum). In some embodiments, the term “barley” refers to barley of the species Hordeum vulgare.

As referred to herein, “wheat” should be understood to include plants of the genus Triticum. Thus, the term “wheat” encompasses diploid wheat, tetraploid wheat and hexaploid wheat. In some embodiments, the wheat plant may be a cultivated species of wheat including, for example, Triticum aestivum, Triticum durum, Triticum monococcum or Triticum spelta. In some embodiments, the term “wheat” refers to wheat of the species Triticum aestivum.

As referred to herein, “rice” includes several members of the genus Oryza including the species Oryza sativa and Oryza glaberrima. The term “rice” thus encompasses rice cultivars such as japonica or sinica varieties, indica varieties and javonica varieties. In some embodiments, the term “rice” refers to rice of the species Oryza sativa.

In some embodiments, the present invention also contemplates the use of other monocotyledonous plants, such as other non-cereal plants of the Poales, specifically including pasture grasses such as Lolium spp.

In some embodiments, the term “plant” may include a plant of the genus Plantago. In some embodiments, a plant of the genus Plantago may include a plant from any one of the more than 200 Plantago species including, for example, a plant of a species selected from Plantago ovata, Plantago patagonica, Plantago hawaiensis, Plantago princeps, Plantago amplexicaulis, Plantago hookeriana, Plantago aristata, Plantago asiatica, Plantago lanceolata, Plantago rhodosperma, Plantago cordata, Plantago major, Plantago rigida, Plantago coronopus, Plantago rugelii, Plantago maritima, Plantago cretica, Plantago media, Plantago elongata, Plantago erecta, Plantago eriopoda, Plantago moorei, Plantago subnuda, Plantago nivalis or Plantago nubicola.

In some embodiments, the term “plant” may include a tree species. Examples of tree species include Poplar spp., Pinus spp. and other species used in the pulp and paper industries.

The cell contemplated by the present invention may be, for example, an isolated cell or a cell comprising part of a multicellular structure (as defined hereafter).

As set out above, the present invention is predicated, in part, on modulating the level and/or activity of a XynS xylan synthase in a cell.

In some embodiments, the term “XynS xylan synthase” should be understood to encompass any protein which catalyses the synthesis of xylan and/or at least catalyses the polymerisation of (1,4)-linked β-D-xylopyranosyl residues and/or oligosaccharides.

In some embodiments, the XynS xylan synthase comprises an amino acid sequence having at least 50% amino acid sequence identity to one or more of:
GenBank accession number AAR29963;
GenBank accession number AAR29965;
SEQ ID NO: 1;
SEQ ID NO: 3; and/or
SEQ ID NO: 5.

In some embodiments the XynS xylan synthase comprises an amino acid sequence having at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99% at least 99.5% or 100% amino acid sequence identity to one or more of GenBank accession number AAR29963, GenBank accession number AAR29965, SEQ ID NO: 1, SEQ ID NO: 3 and/or SEQ ID NO: 5.

When comparing amino acid sequences, the compared sequences should be compared over a comparison window of at least 100 amino acid residues, at least 200 amino acid residues, at least 500 amino acid residues, at least 800 amino acid residues or over the full length of one or more of GenBank accession number AAR29963, GenBank accession number AAR29965, SEQ ID NO: 1, SEQ ID NO: 3 and/or SEQ ID NO: 5. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms such as the BLAST family of programs as, for example, disclosed by Altschul et al. (Nucl. Acids Res. 25: 3389-3402, 1997). A detailed discussion of sequence analysis can be found in Unit 19. 3 of Ausubel et al. (Current Protocols in Molecular Biology, John Wiley & Sons Inc, 1994-1998, Chapter 15, 1998).

In some embodiments, the XynS xylan synthase comprises one or more of:
(i) a polypeptide comprising an amino acid sequence selected from the list consisting of GenBank accession numbers: AAR29963, XP_{—}003559270, NP_{—}001051648, XP_{—}002463687 or NP_{—}001104955;
(ii) a polypeptide comprising having an amino acid sequence selected from the list consisting of GenBank accession numbers: AAR29965, XP_{—}003569818, NP_{—}001044252, XP_{—}002456361, NP_{—}001105236 or CBH32503;
(iii) a polypeptide comprising the amino acid sequence set forth in SEQ ID NO: 1;
(iv) a polypeptide comprising the amino acid sequence set forth in SEQ ID NO: 3;
(v) a polypeptide comprising the amino acid sequence set forth in SEQ ID NO: 5; and/or
(vi) a polypeptide comprising an ortholog of a polypeptide defined at any of (i) to (v) above.

As referred to herein, modulation of the “level” of a XynS xylan synthase in a cell should be understood to include modulation of the abundance of a XynS nucleic acid transcript and/or modulation of the abundance of a XynS xylan synthase polypeptide in the cell.

Modulation of the “activity” of a XynS xylan synthase should be understood to include modulation of the total activity, specific activity, half-life and/or stability of the XynS xylan synthase in the cell.

By “modulating” with regard to the level and/or activity of the XynS xylan synthase is intended decreasing or increasing the level and/or activity of XynS xylan synthase in the cell.

By “decreasing” is intended, for example, a 1%, 5%, 10%, 20%, 30%, 40%, 50%, 60%, 70%, 80%, 90%, 91%, 92%, 93%, 94%, 95%, 96%, 97%, 98%, 99%, 100% reduction in the level and/or activity of XynS xylan synthase in the cell relative to a wild type of the cell.

By “increasing” is intended, for example, a 1%, 5%, 10%, 20%, 30%, 40%, 50%, 60%, 70%, 80%, 90%, 100%, 2-fold, 5-fold, 10-fold, 20 fold, 50-fold, 100-fold increase in the level and/or activity of XynS xylan synthase in the cell relative to a wild type of the cell.

“Modulating” also includes introducing a XynS xylan synthase into a cell which does not normally express the introduced enzyme, or the substantially complete inhibition of XynS xylan synthase activity in a cell that normally has such activity.

The methods of the present invention contemplate any means known in the art by which the level and/or activity of a XynS xylan synthase in a cell may be modulated. This includes methods such as the application of agents which modulate XynS xylan synthase activity in a cell, such as the application of a XynS xylan synthase agonist or antagonist; the application of agents which mimic XynS xylan synthase activity in a cell; modulating the expression of a XynS nucleic acid which encodes XynS xylan synthase in the cell; or effecting the expression of an altered or mutated XynS nucleic acid in a cell such that a XynS xylan synthase with increased or decreased specific activity, half-life and/or stability is expressed by the cell.

In some embodiments, the level and/or activity of a XynS xylan synthase is modulated by modulating the expression of a XynS nucleic acid in the cell.

Therefore, in a second aspect, the present invention provides a method for modulating the level and/or activity of a XynS xylan synthase in a cell, the method comprising modulating the expression of a XynS nucleic acid in the cell.

As used herein, the term “XynS nucleic acid” should be understood to include to a nucleic acid molecule which encodes a XynS xylan synthase as defined herein.

In some embodiments, the XynS nucleic acid comprises one or more of:
a nucleotide sequence which comprises at least 50% nucleotide sequence identity to one or more of: the nucleotide sequence set forth in GenBank accession number AY483151, GenBank accession number AY483153, SEQ ID NO: 2, SEQ ID NO: 4 and/or SEQ ID NO: 6; and/or
a nucleotide sequence which hybridises to a nucleic acid molecule comprising the nucleotide sequence set forth in one or more of: GenBank accession number AY483151, GenBank accession number AY483153, SEQ ID NO: 2, SEQ ID NO: 4 and/or SEQ ID NO: 6 under stringent conditions.

In some embodiments the XynS nucleic acid comprises at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99% at least 99.5% or 100% sequence identity to one or more of: GenBank accession number AY483151, GenBank accession number AY483153, SEQ ID NO: 2, SEQ ID NO: 4 and/or SEQ ID NO: 6.

When comparing nucleic acid sequences to one or more of GenBank accession number AY483151, GenBank accession number AY483153, SEQ ID NO: 2, SEQ ID NO: 4 and/or SEQ ID NO: 6 to calculate a percentage identity, the compared nucleic acid sequences should be compared over a comparison window of: at least 100 nucleotide residues, at least 300 nucleotide residues, at least 600 nucleotide residues, at least 1200 nucleotide residues, at least 2400 nucleotide residues, the protein encoding region of the compared sequences and/or over the full length of the compared sequences. The comparison window may comprise additions or deletions (ie. gaps) of about 20% or less as compared to the reference sequence (which does not comprise additions or deletions) for optimal alignment of the two sequences. Optimal alignment of sequences for aligning a comparison window may be conducted by computerized implementations of algorithms such as the BLAST family of programs as, for example, disclosed by Altschul et al. (Nucl. Acids Res. 25: 3389-3402, 1997). A detailed discussion of sequence analysis can be found in Unit 19. 3 of Ausubel et al. (Current Protocols in Molecular Biology, John Wiley & Sons Inc, 1994-1998, Chapter 15, 1998).

As set out above, in some embodiments, the XynS nucleic acid may also comprise a nucleic acid that hybridises to a nucleic acid molecule comprising the nucleotide sequence set forth in one or more of GenBank accession number AY483151, GenBank accession number AY483153, SEQ ID NO: 2, SEQ ID NO: 4 and/or SEQ ID NO: 6 under stringent conditions. As used herein, “stringent” hybridisation conditions will be those in which the salt concentration is less than about 1.5 M Na ion, typically about 0.01 to 1.0 M Na ion concentration (or other salts) at pH 7.0 to 8.3 and the temperature is at least 300° C. Stringent conditions may also be achieved with the addition of destabilizing agents such as formamide. Stringent hybridisation conditions may be low stringency conditions, medium stringency conditions or high stringency conditions. Exemplary low stringency conditions include hybridisation with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary medium stringency conditions include hybridisation in 40 to 45% formamide, 1.0 M NaCl, 1% SDS at 37° C., and a wash in 0.5× to 1×SSC at 55° C. to 600° C. Exemplary high stringency conditions include hybridisation in 50% formamide, 1 M NaCl, 1% SDS at 37° C., and a wash in 0.1×SSC at 60° C. to 65° C. Optionally, wash buffers may comprise about 0.1% to about 1% SDS. Duration of hybridization is generally less than about 24 hours, usually about 4 to about 12 hours.

Specificity of hybridisation is also a function of post-hybridization washes, and is influenced by the ionic strength and temperature of the final wash solution. For DNA-DNA hybrids, the Tm can be approximated from the equation: Tₘ=81.5° C.+16.6 (log M)+0.41 (% GQ−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization, and/or wash conditions can be adjusted to hybridize to sequences of different degrees of complementarity. For example, sequences with >90% identity can be hybridised by decreasing the Tm by about 10° C. Generally, stringent conditions are selected to be lower than the thermal melting point (Tm) for the specific sequence and its complement at a defined ionic strength and pH. For example, high stringency conditions can utilize a hybridization and/or wash at, for example, 1, 2, 3, 4 or 5° C. lower than the thermal melting point (Tm); medium stringency conditions can utilize a hybridization and/or wash at, for example, 6, 7, 8, 9, or 10° C. lower than the thermal melting point (Tm); low stringency conditions can utilize a hybridization and/or wash at, for example, 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution), the SSC concentration may be increased so that a higher temperature can be used. An extensive guide to the hybridization of nucleic acids is found in Tijssen (Laboratory Techniques in Biochemistry and Molecular Biology-Hybridization with Nucleic acid Probes, Pt I, Chapter 2, Elsevier, New York, 1993), Ausubel et al., eds. (Current Protocols in Molecular Biology, Chapter 2, Greene Publishing and Wiley-Interscience, New York, 1995) and Sambrook et al. (Molecular Cloning: A Laboratory Manual, 2nd ed., Cold Spring Harbor Laboratory Press, Plainview, N.Y., 1989).

The XynS nucleic acids contemplated by the present invention may also comprise one or more non-translated regions such as 3′ and 5′ untranslated regions and/or introns. For example, the XynS nucleic acids contemplated by the present invention may comprise, for example, mRNA sequences, cDNA sequences or genomic nucleotide sequences.

In some embodiments, the XynS nucleic acid comprises one or more of:
(i) a nucleic acid comprising a nucleotide sequence selected from the list consisting of GenBank accession numbers: AY483151, XM_{—}003559222, NM_{—}001058183, XM_{—}002463642 or NM_{—}001111485;
(ii) a nucleic acid comprising a nucleotide sequence selected from the list consisting of GenBank accession numbers: AY483153, XM_{—}003569770, NM_{—}001050787, XM_{—}002456316 or NM_{—}001111766;
(iii) a nucleic acid comprising the nucleotide sequence set forth in SEQ ID NO: 2;
(iv) a nucleic acid comprising the nucleotide sequence set forth in SEQ ID NO: 4;
(v) a nucleic acid comprising the nucleotide sequence set forth in SEQ ID NO: 6; and/or
(vi) a nucleic acid comprising a nucleotide sequence which encodes an ortholog of a polypeptide encoded by the nucleotide sequence defined at any of (i) to (v) above.

As mentioned above, the present invention provides methods for modulating the expression of a XynS nucleic acid in a cell. The present invention contemplates any method by which the expression of a XynS nucleic acid in a cell may be modulated.

The term “modulating” with regard to the expression of a XynS nucleic acid is generally intended to refer to decreasing or increasing the transcription and/or translation of a XynS nucleic acid.

By “decreasing” is intended, for example, a 1%, 5%, 10%, 20%, 30%, 40%, 50%, 60%, 70%, 80%, 90%, 91%, 92%, 93%, 94%, 95%, 96%, 97%, 98%, 99%, 100% reduction in the transcription and/or translation of a XynS nucleic acid in a cell relative to a wild type of the cell.

By “increasing” is intended, for example a 1%, 5%, 10%, 20%, 30%, 40%, 50%, 60%, 70%, 80%, 90%, 100%, 2-fold, 5-fold, 10-fold, 20-fold, 50-fold, 100-fold or greater increase in the transcription and/or translation of a XynS nucleic acid in a cell relative to a wild type of the cell.

In some embodiments, “modulating” may also comprise introducing expression of a XynS nucleic acid not normally found in a particular cell; or the substantially complete inhibition (eg. knockout) of expression of a XynS nucleic acid in a cell that normally has such activity.

Methods for modulating the expression of a particular nucleic acid molecule in a cell are known in the art and the present invention contemplates any such method. Exemplary methods for modulating the expression of a XynS nucleic acid include: genetic modification of the cell to upregulate or downregulate endogenous XynS nucleic acid expression; genetic modification by transformation with a XynS nucleic acid; administration of a nucleic acid molecule to the cell which modulates expression of an endogenous XynS nucleic acid in the cell; and the like.

In some embodiments, the expression of a XynS nucleic acid is modulated by genetic modification of the cell. The term “genetically modified”, as used herein, should be understood to include any genetic modification that effects an alteration in the expression of a XynS nucleic acid in the genetically modified cell relative to a non-genetically modified form of the cell. Exemplary types of genetic modification contemplated herein include: random mutagenesis such as transposon, chemical, radiation (including UV and/or X-ray) or phage mutagenesis together with selection of mutants which overexpress or underexpress an endogenous XynS nucleic acid; transient or stable introduction of one or more nucleic acid molecules into a cell which direct the expression and/or overexpression of XynS nucleic acid in the cell; site-directed mutagenesis of an endogenous XynS nucleic acid; introduction of one or more nucleic acid molecules which inhibit the expression of an endogenous XynS nucleic acid in the cell, eg. a cosuppression construct or an RNAi construct; and the like.

In some embodiments, the genetic modification comprises the introduction of a nucleic acid into a cell of interest.

The nucleic acid may be introduced using any method known in the art which is suitable for the cell type being used, for example, those described in Sambrook and Russell (Molecular Cloning—A Laboratory Manual, 3rd Ed., Cold Spring Harbor Laboratory Press, 2000).

In some embodiments wherein the cell is a plant cell, suitable methods for introduction of a nucleic acid molecule may include: Agrobacterium-mediated transformation, microprojectile bombardment based transformation methods and direct DNA uptake based methods. For example, Roa-Rodriguez et al. (Agrobacterium-mediated transformation of plants, 3rd Ed. CAMBIA Intellectual Property Resource, Canberra, Australia, 2003) review a wide array of suitable Agrobacterium-mediated plant transformation methods for a wide range of plant species. Microprojectile bombardment may also be used to transform plant tissue and methods for the transformation of plants, particularly cereal plants, and examples of such methods are reviewed by Casas et al. (Plant Breeding Rev. 13: 235-264, 1995). Direct DNA uptake transformation protocols such as protoplast transformation and electroporation are exemplified in Galbraith et al. (eds.), (Methods in Cell Biology Vol. 50, Academic Press, San Diego, 1995). In addition to the methods mentioned above, a range of other transformation protocols may also be used.

Examples include infiltration, electroporation of cells and tissues, electroporation of embryos, microinjection, pollen-tube pathway, silicon carbide- and liposome mediated transformation. Examples of such methods are reviewed by Rakoczy-Trojanowska (Cell. Mol. Biol. Lett. 7: 849-858, 2002). A range of other suitable plant transformation methods may also be evident to those of skill in the art and the present invention should not be considered limited to the specific transformation methods noted above.

The introduced nucleic acid may be single stranded or double stranded. The nucleic acid may be transcribed into mRNA and translated into a XynS xylan synthase or another protein; may encode for a non-translated RNA such as an RNAi construct, cosuppression construct, antisense RNA, tRNA, miRNA, siRNA, ntRNA and the like; or may act directly in the cell. The introduced nucleic acid may be an unmodified DNA or RNA or a modified DNA or RNA which may include modifications to the nucleotide bases, sugar or phosphate backbones but which retain functional equivalency to a nucleic acid. The introduced nucleic acid may optionally be replicated in the cell; integrated into a chromosome or any extrachromosomal elements of the cell; and/or transcribed by the cell. Also, the introduced nucleic acid may be either homologous or heterologous with respect to the host cell. That is, the introduced nucleic acid may be derived from a cell of the same species as the genetically modified cell (ie. homologous) or the introduced nucleic may be derived from a different species (ie. heterologous). The transgene may also be a synthetic transgene.

In some embodiments, the present invention contemplates increasing the level of xylan produced by a cell, by expressing, overexpressing or introducing a XynS nucleic acid into the cell.

In further embodiments, the present invention also provides methods for down-regulating expression of a xylan synthase in a cell.

For example, the identification of XynS nucleic acids encoding xylan synthases facilitates methods such as knockout or knockdown of a xylan synthase in a cell using methods such as:
insertional mutagenesis of a XynS nucleic acid in a cell, including knockout or knockdown of a XynS nucleic acid in a cell, by homologous recombination with a knockout construct (for an example of targeted gene disruption in plants see Terada et al., Nat. Biotechnol. 20: 1030-1034, 2002);
post-transcriptional gene silencing (PTGS) or RNAi of a XynS nucleic acid in a cell (for review of PTGS and RNAi see Sharp, Genes Dev. 15(5): 485-490, 2001; and Harmon, Nature 418: 244-51, 2002);
transformation of a cell with an antisense construct directed against a XynS nucleic acid (for examples of antisense suppression in plants see van der Krol et al., Nature 333: 866-869; van der Krol et al., BioTechniques 6: 958-967; and van der Krol et al., Gen. Genet. 220: 204-212);
transformation of a cell with a co-suppression construct directed against a XynS nucleic acid (for an example of co-suppression in plants see van der Krol et al., Plant Cell 2(4): 291-299);
transformation of a cell with a construct encoding a double stranded RNA directed against a XynS nucleic acid (for an example of dsRNA mediated gene silencing see Waterhouse et al., Proc. Natl. Acad. Sci. USA 95: 13959-13964, 1998);
transformation of a cell with a construct encoding an siRNA or hairpin RNA directed against a XynS nucleic acid (for an example of siRNA or hairpin RNA mediated gene silencing in plants see Lu et al., Nucl. Acids Res. 32(21): e171, 2004); and/or
insertion of a miRNA target sequence such that it is in operable connection with XynS nucleic acid (for an example of miRNA mediated gene silencing see Brown et al., Blood 110(13): 4144-4152, 2007).

The present invention also facilitates the downregulation of a XynS nucleic acid in a cell via the use of synthetic oligonucleotides such as siRNAs or microRNAs directed against a XynS nucleic acid which are administered to a cell. For examples of synthetic siRNA mediated silencing see Caplen et al. (Proc. Natl. Acad. Sci. USA 98: 9742-9747, 2001); Elbashir et al. (Genes Dev. 15: 188-200, 2001); and Elbashir et al. (EMBO J. 20: 6877-6888, 2001).

In addition to the examples above, the introduced nucleic acid may also comprise a nucleotide sequence which is not directly related to a XynS nucleic acid but, nonetheless, may directly or indirectly modulate the expression of XynS nucleic acid in a cell. Examples include nucleic acid molecules that encode transcription factors or other proteins which promote or suppress the expression of an endogenous XynS nucleic acid molecule in a cell; and other non-translated RNAs which directly or indirectly promote or suppress endogenous xylan synthase expression and the like.

In order to effect expression of an introduced nucleic acid in a genetically modified cell, where appropriate, the introduced nucleic acid may be operably connected to one or more control sequences. The term “control sequences” should be understood to include any nucleotide sequences which are necessary or advantageous for the transcription, translation and or post-translational modification of the operably connected nucleic acid or the transcript or protein encoded thereby. Each control sequence may be native or foreign to the operably connected nucleic acid. The control sequences may include, but are not limited to, a leader, polyadenylation sequence, propeptide encoding sequence, promoter, enhancer or upstream activating sequence, signal peptide encoding sequence, and transcription terminator. Typically, a control sequence at least includes a promoter.

The term “promoter” as used herein, describes any nucleic acid which confers, activates or enhances expression of a nucleic acid molecule in a cell. Promoters are generally positioned 5′ (upstream) to the genes that they control. In the construction of heterologous promoter/gene combinations, it may be desirable to position the promoter at a distance from the gene transcription start site that is approximately the same as the distance between that promoter and the gene it controls in its natural setting, ie. the gene from which the promoter is derived. In some embodiments, some variation in this distance can be accommodated without loss of promoter function.

A promoter may regulate the expression of an operably connected nucleotide sequence constitutively, or differentially with respect to the cell, tissue, organ or developmental stage at which expression occurs, in response to external stimuli such as physiological stresses, pathogens, or metal ions, amongst others, or in response to one or more transcriptional activators. As such, the promoter used in accordance with the methods of the present invention may include a constitutive promoter, an inducible promoter, a tissue-specific promoter or an activatable promoter.

The present invention contemplates the use of any promoter which is active in a cell of interest. As such, a wide array of promoters which are active in any of bacteria, fungi, animal cells or plant cells would be readily ascertained by one of ordinary skill in the art. However, in some embodiments, plant cells are used. In these embodiments, plant-active constitutive, inducible, tissue-specific or activatable promoters are typically used.

Plant constitutive promoters typically direct expression in nearly all tissues of a plant and are largely independent of environmental and developmental factors. Examples of constitutive promoters that may be used in accordance with the present invention include plant viral derived promoters such as the Cauliflower Mosaic Virus 35S and 19S (CaMV 35S and CaMV 19S) promoters; bacterial plant pathogen derived promoters such as opine promoters derived from Agrobacterium spp., eg. the Agrobacterium-derived nopaline synthase (nos) promoter; and plant-derived promoters such as the rubisco small subunit gene (rbcS) promoter, the plant ubiquitin promoter (Pubi) and the rice actin promoter (Pact).

“Inducible” promoters include, but are not limited to, chemically inducible promoters and physically inducible promoters. Chemically inducible promoters include promoters which have activity that is regulated by chemical compounds such as alcohols, antibiotics, steroids, metal ions or other compounds. Examples of chemically inducible promoters include: alcohol regulated promoters (eg. see European Patent 637339); tetracycline regulated promoters (eg. see U.S. Pat. No. 5,851,796 and U.S. Pat. No. 5,464,758); steroid responsive promoters such as glucocorticoid receptor promoters (eg. see U.S. Pat. No. 5,512,483), estrogen receptor promoters (eg. see European Patent Application 1 232 273), ecdysone receptor promoters (eg. see U.S. Pat. No. 6,379,945) and the like; metal-responsive promoters such as metallothionein promoters (eg. see U.S. Pat. No. 4,940,661, U.S. Pat. No. 4,579,821 and U.S. Pat. No. 4,601,978); and pathogenesis related promoters such as chitinase or lysozyme promoters (eg. see U.S. Pat. No. 5,654,414) or PR protein promoters (eg. see U.S. Pat. No. 5,689,044, U.S. Pat. No. 5,789,214, Australian Patent 708850, U.S. Pat. No. 6,429,362).

The inducible promoter may also be a physically regulated promoter which is regulated by non-chemical environmental factors such as temperature (both heat and cold), light and the like. Examples of physically regulated promoters include heat shock promoters (eg. see U.S. Pat. No. 5,447,858, Australian Patent 732872, Canadian Patent Application 1324097); cold inducible promoters (eg. see U.S. Pat. No. 6,479,260, U.S. Pat. No. 6,084,08, U.S. Pat. No. 6,184,443 and U.S. Pat. No. 5,847,102); light inducible promoters (eg. see U.S. Pat. No. 5,750,385 and Canadian Patent 132 1563); light repressible promoters (eg. see New Zealand Patent 508103 and U.S. Pat. No. 5,639,952).

“Tissue specific promoters” include promoters which are preferentially or specifically expressed in one or more specific cells, tissues or organs in an organism and/or one or more developmental stages of the organism. It should be understood that a tissue specific promoter may, in some cases, also be inducible.

Examples of plant tissue specific promoters include: root specific promoters such as those described in US Patent Application 2001047525; fruit specific promoters including ovary specific and receptacle tissue specific promoters such as those described in European Patent 316 441, U.S. Pat. No. 5,753,475 and European Patent Application 973 922; and seed specific promoters such as those described in Australian Patent 612326 and European Patent application 0 781 849 and Australian Patent 746032.

In some embodiments, the tissue specific promoter is a seed and/or grain specific promoter. Exemplary seed or grain specific promoters include puroindoline-b gene promoters (for example see Digeon et al, Plant Mol. Biol. 39: 1101-1112, 1999); Pbf gene promoters (for example see Mena et al., Plant J. 16: 53-62, 1998); GS1-2 gene promoters (for example see Muhitch et al, Plant Sci. 163: 865-872, 2002); glutelin or Gt1 gene promoters (for example see Okita et al, J. Biol. Chem. 264: 12573-12581, 1989; Zheng et al, Plant J. 4: 357-366, 1993; Sindhu et al, Plant Sci. 130: 189-196, 1997; Nandi et al, Plant Sci. 163: 713-722, 2002); Hor2-4 gene promoters (for example see Knudsen and Müller, Planta 195: 330-336, 1991; Patel et al, Mol. Breeding 6: 113-123, 2000; Wong et al, Proc. Natl. Acad. Sci. USA 99: 16325-16330, 2002); lipoxygenase 1 gene promoters (for example see Rouster et al, Plant J. 15: 435-440, 1998); CM26 gene promoters (for example see Leah et al, Plant J. 6: 579-589, 1994); Glu-Dl-1 gene promoters (for example see Lamacchia et al, J. Exp. Bot. 52: 243-250, 2001; Zhang et al, Theor. Appl. Genet. 106: 1139-1146, 2003); Hor3-1 gene promoters (for example see Sörensen et al, Mol Gen. Genet. 250: 750-760, 1996; Horvath et al, Proc. Natl. Acad. Sci. USA 97: 1914-1919, 2000), Waxy (Wx) gene promoters (for example see Yao et al, Acta Phytophysiol. Sin. 22: 431-436, 1996; Terada et al, Plant Cell Physiol. 41: 881-888, 2000; Liu et al, Transgenic Res. 12: 71-82, 2003) and the oat globulin (AsGLO) promoter (see Vickers et al., Plant Mol. Biol. 62: 195-214, 2006).

The promoter may also be a promoter that is activatable by one or more defined transcriptional activators, referred to herein as an “activatable promoter”. For example, the activatable promoter may comprise a minimal promoter operably connected to an Upstream Activating Sequence (UAS), which comprises, inter alia, a DNA binding site for one or more transcriptional activators.

As referred to herein the term “minimal promoter” should be understood to include any promoter that incorporates at least an RNA polymerase binding site and, preferably a TATA box and transcription initiation site and/or one or more CAAT boxes. When the cell is a plant cell, the minimal promoter may be derived from, for example, the Cauliflower Mosaic Virus 35S (CaMV 35S) promoter. The CaMV 35S derived minimal promoter may comprise, for example, a sequence that corresponds to positions −90 to +1 (the transcription initiation site) of the CaMV 35S promoter (also referred to as a −90 CaMV 35S minimal promoter), −60 to +1 of the CaMV 35S promoter (also referred to as a −60 CaMV 35S minimal promoter) or −45 to +1 of the CaMV 35S promoter (also referred to as a −45 CaMV 35S minimal promoter).

As set out above, the activatable promoter may comprise a minimal promoter fused to an Upstream Activating Sequence (UAS). The UAS may be any sequence that can bind a transcriptional activator to activate the minimal promoter. Exemplary transcriptional activators include, for example: yeast derived transcription activators such as Gal4, Pdrl, Gcn4 and Ace1; the viral derived transcription activator, VP16; Hap1 (Hach et al, J Biol Chem 278: 248-254, 2000); Gafl (Hoe et al., Gene 215(2): 319-328, 1998); E2F (Albani et al, J Biol Chem 275: 19258-19267, 2000); HAND2 (Dai and Cserjesi, J Biol Chem 277: 12604-12612, 2002); NRF-1 and EWG (Herzig et al., J Cell Sci 113: 4263-4273, 2000); P/CAF (Itoh et al, Nucl Acids Res 28: 4291-4298, 2000); Maf A (Kataoka et al, J Biol Chem 277: 49903-49910, 2002); human activating transcription factor 4 (Liang and Hai, J Biol Chem 272: 24088-24095, 1997); BcIlO (Liu et al, Biochem Biophys Res Comm 320(1): 1-6, 2004); CREB-H (Omori et al, Nucl Acids Res 29: 2154-2162, 2001); ARR1 and ARR2 (Sakai et al, Plant J 24(6): 703-711, 2000); Fos (Szuts and Bienz, Proc Natl Acad Sci USA 97: 5351-5356, 2000); HSF4 (Tanabe et al., J Biol Chem 274: 27845-27856, 1999); MAML1 (Wu et al, Nat Genet 26: 484-489, 2000).

As mentioned above, the control sequences may also include a terminator. The term “terminator” refers to a DNA sequence at the end of a transcriptional unit which signals termination of transcription. Terminators are 3′-non-translated DNA sequences generally containing a polyadenylation signal, which facilitates the addition of polyadenylate sequences to the 3′-end of a primary transcript. As with promoter sequences, the terminator may be any terminator sequence which is operable in the cells, tissues or organs in which it is intended to be used. Examples of suitable terminator sequences which may be useful in plant cells include: the nopaline synthase (nos) terminator, the CaMV 35S terminator, the octopine synthase (ocs) terminator, potato proteinase inhibitor gene (pin) terminators, such as the pinII and pinIII terminators and the like.

Modulating the level of xylan in a cell, by modulating the level and/or activity of a XynS xylan synthase in the cell, has several industrial applications, non-limiting examples of which are set out below:

For example, soluble or solubilized xylans are known to form viscous solutions. The viscosity-generating properties of xylans are critical determinants in many aspects of cereal processing. For example, incompletely degraded xylans from malted barley and cereal adjuncts can contribute to wort and beer viscosity and are associated with problems in wort separation and beer filtration. Therefore, in some embodiments, the present invention may be applied to reduce the level of xylan in cereal grain, by reducing the level and/or activity of a XynS xylan synthase in one or more cells of the cereal grain, to increase its suitability for beer production.

Xylans are also considered to have antinutritive effects in monogastric animals such as pigs and poultry. The “antinutritive” effects have been attributed to the increased viscosity of gut contents, which slows both the diffusion of digestive enzymes and the absorption of degradative products of enzyme action. This, in turn, leads to slower growth rates. Moreover, in dietary formulations for poultry, high xylan concentrations are associated with ‘sticky’ faeces, which are indicative of the poor digestibility of the xylan and which may present major handling and hygiene problems for producers. Therefore, in some embodiments, the present invention may be applied to reducing the level of xylan in one or more cells of a plant used for animal feed, to improve the suitability of the plant as animal feed.

However, xylans are important components of dietary fibre in human and animal diets. As used herein, the term “dietary fibre” should be understood to include edible carbohydrate polymers occurring in food that are not hydrolysed by endogenous enzymes in the small intestine. In at least human diets, dietary fibres promote beneficial physiological effects including general bowel health, laxation, blood cholesterol attenuation, and/or blood glucose attenuation. Dietary fibre has also been linked to reduced risk of contracting serious human diseases, including type II diabetes, colorectal cancer, cardiovascular disease and certain inflammatory diseases, such as emphysema and asthma.

Humans and monogastric animals produce no enzymes that degrade xylans, although there are indications that some depolymerisation occurs in the stomach and small intestine. By comparison, the soluble xylans and other non-starchy polysaccharides are readily fermented by colonic micro-organisms and make a small contribution to digestible energy. In contrast to their antinutritive effects in monogastric animals, xylans at high concentrations in humans have beneficial effects, especially for non-insulin-dependent diabetics, by flattening glucose and insulin responses that follow a meal. High concentrations of xylans (eg. 20% w/v) in food have also been implicated in the reduction of serum cholesterol concentrations, by lowering the uptake of dietary cholesterol or resorption of bile acids from the intestine (see Belobrajdic et al., Brit J Nutrition 107: 1274-1282, 2012).

Therefore, in some embodiments, the present invention may be applied to increasing the dietary fibre content of an edible plant or edible plant part, by increasing the level of xylan in the plant, or part thereof. In some embodiments, the edible plant or edible part of a plant is a cereal crop plant or part thereof.

Xylans, in common with a number of other polysaccharides, may also modify immunological responses in humans by a process that is mediated through binding to receptors on cells of the reticuloendothelial system (leucocytes and macrophages). In addition, they may have the capacity to activate the proteins of the human complement pathway, a system that is invoked as a first line of defence before circulating antibodies are produced.

Heteroxylans are also important in the baking industry, where they are known to affect such parameters as loaf volume but also crumb quality. Depending on the baking system, it might be desirable to increase or decrease the levels of heteroxylans in wheat flour or other cereal flours used for baking.

In paper manufacture, the content of xylans in comparison to cellulose contents can affect paper quality. Thus, it might be desirable to increase or decrease xylan content in these applications.

The present invention also facilitates the production of xylan in a recombinant expression system. For example, xylan may be recombinantly produced by introducing a XynS nucleic acid under the control of a promoter, into a cell, wherein the cell subsequently expresses a XynS xylan synthase and produces xylan.

A vast array of recombinant expression systems that may be used to express a XynS nucleic acid are known in the art. Exemplary recombinant expression systems include:
bacterial expression systems such as E. coli expression systems (for example as reviewed in Baneyx, Curr. Opin. Biotechnol. 10: 411-421, 1999; eg. see also Gene expression in recombinant microorganisms, Smith (Ed.), Marcel Dekker, Inc. New York, 1994; and Protein Expression Technologies: Current Status and Future Trends, Baneyx (Ed.), Chapters 2 and 3, Horizon Bioscience, Norwich, UK, 2004), Bacillus spp. expression systems (eg. see Protein Expression Technologies: Current Status and Future Trends, supra, chapter 4) and Streptomyces spp. expression systems (eg. see Practical Streptomyces Genetics, Kieser et al., (Eds.), Chapter 17, John Innes Foundation, Norwich, UK, 2000);
fungal expression systems including yeast expression systems such as Saccharomyces spp. (including Saccharomyces cerevisiae), Schizosaccharomyces pombe, Hansenula polymorpha and Pichia spp. expression systems and filamentous fungi expression systems (eg. see Protein Expression Technologies: Current Status and Future Trends, supra, chapters 5, 6 and 7; Buckholz and Gleeson, Bio/Technology 9(11): 1067-1072, 1991; Cregg et al, Mol. Biotechnol. 16(1): 23-52, 2000; Cereghino and Cregg, FEMS Microbiology Reviews 24: 45-66, 2000; Cregg et al., Bio/Technology 11: 905-910, 1993);
mammalian cell expression systems including Chinese Hamster Ovary (CHO) cell based expression systems (eg. see Protein Expression Technologies: Current Status and Future Trends, supra, chapter 9);
insect cell cultures including baculovirus expression systems (eg. see Protein Expression Technologies: Current Status and Future Trends, supra, chapter 8; Kost and Condreay, Curr. Opin. Biotechnol. 10: 428-433, 1999; Baculovirus Expression Vectors: A Laboratory Manual WH Freeman & Co., New York, 1992; and The Baculovirus Expression System: A Laboratory Manual, Chapman & Hall, London, 1992);
plant cell expression systems such as tobacco, soybean, rice and tomato cell expression systems (eg. see review of Hellwig et al, Nat Biotechnol 22: 1415-1422, 2004);
and the like.

Therefore, in a third aspect, the present invention provides a method for producing xylan, the method comprising transforming a cell with an isolated XynS nucleic acid and allowing the cell to express the isolated XynS nucleic acid.

In some embodiments, the cell is a cell from a recombinant expression system as hereinbefore defined.

In some embodiments, the cell is a plant cell, a monocot plant cell, a Poales plant cell and/or a cereal crop plant cell.

In a fourth aspect, the present invention also provides xylan produced according to the method of the third aspect of the invention.

In a fifth aspect, the present invention also provides a cell comprising:
modulated level and/or activity of XynS xylan synthase relative to a wild type cell of the same taxon; and/or
modulated expression of a XynS nucleic acid relative to a wild type cell of the same taxon.

In some embodiments of the fifth aspect of the invention, the XynS xylan synthase comprises an amino acid sequence having at least 50% identity to one or more of: GenBank accession number AAR29963; GenBank accession number AAR29965; SEQ ID NO: 1, SEQ ID NO: 3 and/or SEQ ID NO: 5.

In some embodiments of the fifth aspect of the invention the XynS nucleic acid comprises one or more of:
a nucleotide sequence which comprises at least 50% nucleotide sequence identity to one or more of: the nucleotide sequence set forth in GenBank accession number AY483151, GenBank accession number AY483153, SEQ ID NO: 2, SEQ ID NO: 4 and/or SEQ ID NO: 6; and/or
a nucleotide sequence which hybridises to a nucleic acid molecule comprising the nucleotide sequence set forth in one or more of: GenBank accession number AY483151, GenBank accession number AY483153, SEQ ID NO: 2, SEQ ID NO: 4 and/or SEQ ID NO: 6 under stringent conditions.

In some embodiments, the cell further comprises a modulated level of xylan relative to a wild type cell of the same taxon.

In some embodiments, the cell of the fifth aspect of the invention is produced according to the methods of the first or second aspects of the present invention as described herein. In further embodiments, the cell is a plant cell, a monocot plant cell, a Poales plant cell and/or a cereal crop plant cell.

Furthermore, in a sixth aspect, the present invention provides a multicellular structure comprising one or more cells according to the fifth aspect of the invention.

As referred to herein, a “multicellular structure” includes any aggregation of one or more cells. As such, the term “multicellular structure” specifically encompasses tissues, organs, whole organisms and parts thereof. Furthermore, a multicellular structure should also be understood to encompass multicellular aggregations of cultured cells such as colonies, plant calli, suspension cultures and the like.

As mentioned above, in some embodiments of the invention, the cell is a plant cell and as such, the present invention includes a whole plant, plant tissue, plant organ, plant part, plant reproductive material or cultured plant tissue, comprising one or more plant cells according to the sixth aspect of the invention.

In another embodiment, the present invention provides a cereal crop plant comprising one or more cells according to the fifth aspect of the invention.

In a some embodiments, the present invention provides cereal grain comprising one or more cells according to the fifth aspect of the invention.

Therefore, in a seventh aspect, the present invention provides a cereal grain comprising a modulated level of xylan, wherein the grain comprises one or more cells comprising:
a modulated level and/or activity of a XynS xylan synthase; and/or
modulated expression of a XynS nucleic acid.

In some embodiments of the seventh aspect of the invention, the XynS xylan synthase comprises an amino acid sequence having at least 50% identity to one or more of: GenBank accession number AAR29963; GenBank accession number AAR29965; SEQ ID NO: 1, SEQ ID NO: 3 and/or SEQ ID NO: 5.

In some embodiments of the seventh aspect of the invention the XynS nucleic acid comprises one or more of:
a nucleotide sequence which comprises at least 50% nucleotide sequence identity to one or more of: the nucleotide sequence set forth in GenBank accession number AY483151, GenBank accession number AY483153, SEQ ID NO: 2, SEQ ID NO: 4 and/or SEQ ID NO: 6; and/or
a nucleotide sequence which hybridises to a nucleic acid molecule comprising the nucleotide sequence set forth in one or more of: GenBank accession number AY483151, GenBank accession number AY483153, SEQ ID NO: 2, SEQ ID NO: 4 and/or SEQ ID NO: 6 under stringent conditions.

A “cereal grain” as referred to herein should be understood to include the seed of a cereal crop plant as hereinbefore described.

By “modulated” with regard to the level of xylan is intended a decreased or increased level of xylan in and/or produced by the grain relative to a wild type of the grain.

By “decreased” is intended, for example, a 1%, 5%, 10%, 20%, 30%, 40%, 50%, 60%, 70%, 80%, 90%, 91%, 92%, 93%, 94%, 95%, 96%, 97%, 98%, 99%, 100% reduction in the level of xylan relative to a wild type of the grain.

By “increased” is intended, for example, a 1%, 5%, 10%, 20%, 30%, 40%, 50%, 60%, 70%, 80%, 90%, 100%, 2-fold, 5-fold, 10-fold, 20 fold, 50-fold, 100-fold increase in the level of xylan relative to a wild type of the grain.

“Modulating” also includes introducing xylan production into a grain which does not normally produce xylan, or the substantially complete inhibition of xylan production in a grain that normally produces xylan.

In some embodiments wherein the grain is a barley grain, the grain comprises a level of xylan of at least 7%, at least 8%, at least 9%, at least 10%, at least 11%, at least 11.5%, at least 12%, at least 12.5%, at least 13%, at least 13.5%, at least 14% or at least 14.4% on a w/w basis of freeze-dried whole grain.

The grain of the present invention may be milled to produce a flour that may be used, inter alia, for the production of food or animal feed.

Thus, in an eighth aspect, the present invention provides flour comprising flour produced by the milling of the grain of the seventh aspect of the invention.

The flour produced by the milling of the grain of the seventh aspect of the invention may optionally comprise other flour components including flours produced by the milling of other grains. Thus, in some embodiments, flour produced by the milling of the grain of the seventh aspect of the invention may comprise, for example, about 10%, 20%, 30%, 40%, 50%, 60%, 70%, 80%, 90% or 100% by weight of the flour of the eighth aspect of the invention.

As referred to herein “milling” contemplates any method known in the art for milling grain, such as those described by Brennan et al. (Manual of Flour and Husk Milling, Brennan et al. (Eds.), AgriMedia, ISBN: 3-86037-277-7).

In some embodiments, the flour produced by the milling of the grain of the seventh aspect of the invention used in the flour comprises an increased or decreased level of xylan compared to flour produced by the milling of wild type grain.

The “flour produced by the milling of one or more other grains” may be flour produced by milling grain derived from any cereal plant, as hereinbefore defined. This component of the flour of the eighth aspect of the invention may, for example, comprise about 0%, 10%, 20%, 30%, 40%, 50%, 60%, 70%, 80% or 90% by weight of the flour.

In some embodiments, the flour produced by the milling of one or more other grains is wheat flour and, therefore, the flour of the eighth aspect of the invention may be particularly suitable for producing bread, cakes, biscuits and the like.

In a ninth aspect, the present invention provides a genetic construct or vector comprising an isolated XynS nucleic acid as hereinbefore defined, or a complement, reverse complement or fragment thereof.

In some embodiments of the ninth aspect of the invention, the XynS xylan synthase comprises an amino acid sequence having at least 50% identity to one or more of: GenBank accession number AAR29963; GenBank accession number AAR29965; SEQ ID NO: 1, SEQ ID NO: 3 and/or SEQ ID NO: 5.

In some embodiments of the ninth aspect of the invention the XynS nucleic acid comprises one or more of:
a nucleotide sequence which comprises at least 50% nucleotide sequence identity to one or more of: the nucleotide sequence set forth in GenBank accession number AY483151, GenBank accession number AY483153, SEQ ID NO: 2, SEQ ID NO: 4 and/or SEQ ID NO: 6; and/or
a nucleotide sequence which hybridises to a nucleic acid molecule comprising the nucleotide sequence set forth in one or more of: GenBank accession number AY483151, GenBank accession number AY483153, SEQ ID NO: 2, SEQ ID NO: 4 and/or SEQ ID NO: 6 under stringent conditions.

In some embodiments, the vectors or constructs of the ninth aspect of the invention may be used, inter alia, for modulating the level of xylan, the level and/or activity of a xylan synthase, and/or expression of a XynS nucleic acid in accordance with earlier aspects of the invention.

“Isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be isolated because that vector, composition of matter, or particular cell is not the original environment of the polynucleotide. An “isolated” nucleic acid molecule should also be understood to include a synthetic nucleic acid molecule, including those produced by chemical synthesis using known methods in the art or by in-vitro amplification (eg. polymerase chain reaction and the like).

The vectors or constructs may comprise any polyribonucleotide or polydeoxyribonucleotide, which may be unmodified RNA or DNA or modified RNA or DNA. For example, the isolated nucleic acid molecules of the invention may comprise single- and double-stranded DNA, DNA that is a mixture of single- and double-stranded regions, single- and double-stranded RNA, and RNA that is mixture of single- and/or double-stranded regions, hybrid molecules comprising DNA and RNA that may be single-stranded or, more typically, double-stranded or a mixture of single- and double-stranded regions. In addition, the isolated nucleic acid molecules may comprise of triple-stranded regions comprising RNA or DNA or both RNA and DNA. The isolated nucleic acid molecules may also contain one or more modified bases or DNA or RNA backbones modified for stability or for other reasons. “Modified” bases include, for example, tritylated bases and unusual bases such as inosine. A variety of modifications can be made to DNA and RNA; thus the vector or construct may include chemically, enzymatically, or metabolically modified forms.

As set out above, the present invention also provides fragments of a nucleotide sequence. “Fragments” of a nucleotide sequence should be at least 20, 50, 100, 200, 500, 1000, 2000 or 3000 nucleotides (nt) in length.

These fragments have numerous uses that include, but are not limited to, diagnostic probes and primers. Of course, larger fragments, are also useful according to the present invention as are fragments corresponding to most, if not all, of a XynS nucleic acid.

In some embodiments, the fragment may comprise a functional fragment of a XynS nucleic acid. That is, the polynucleotide fragments of the invention may encode a polypeptide having xylan synthase functional activity as defined herein.

In some embodiments, the vector or construct may further comprise one or more of: an origin of replication for one or more hosts; a selectable marker gene which is active in one or more hosts; or one or more control sequences which enable transcription of the isolated nucleic acid molecule in a cell.

“Selectable marker genes” include any nucleotide sequences which, when expressed by a cell, confer a phenotype on the cell that facilitates the identification and/or selection of these transformed cells. A range of nucleotide sequences encoding suitable selectable markers are known in the art. Exemplary nucleotide sequences that encode selectable markers include: antibiotic resistance genes such as ampicillin-resistance genes, tetracycline-resistance genes, kanamycin-resistance genes, the AURI-C gene which confers resistance to the antibiotic aureobasidin A, neomycin phosphotransferase genes (eg. nptI and nptII) and hygromycin phosphotransferase genes (eg. hpt); herbicide resistance genes including glufosinate, phosphinothricin or bialaphos resistance genes such as phosphinothricin acetyl transferase encoding genes (eg. bar), glyphosate resistance genes including 3-enoyl pyruvyl shikimate 5-phosphate synthase encoding genes (eg. aroA), bromyxnil resistance genes including bromyxnil nitrilase encoding genes, sulfonamide resistance genes including dihydropterate synthase encoding genes (eg. sul) and sulfonylurea resistance genes including acetolactate synthase encoding genes; enzyme-encoding reporter genes such as GUS and chloramphenicolacetyltransferase (CAT) encoding genes; fluorescent reporter genes such as the green fluorescent protein-encoding gene; and luminescence-based reporter genes such as the luciferase gene, amongst others.

Furthermore, it should be noted that the selectable marker gene may be a distinct open reading frame in the construct or may be expressed as a fusion protein with the XynS xylan synthase polypeptide.

As set out above, the vector or construct may further comprise a control sequence such as those hereinbefore described.

In some embodiments, the vector or construct is adapted to be at least partially transferred into a plant cell via Agrobacterium-mediated transformation. Accordingly, the vector or construct may comprise left and/or right T-DNA border sequences.

Suitable T-DNA border sequences would be readily ascertained by one of skill in the art. However, the term “T-DNA border sequences” may include substantially homologous and substantially directly repeated nucleotide sequences that delimit a nucleic acid molecule that is transferred from an Agrobacterium sp. cell into a plant cell susceptible to Agrobacterium-mediated transformation. By way of example, reference is made to the paper of Peralta and Ream (Proc. Natl. Acad. Sci. USA, 82(15): 5112-5116, 1985) and the review of Gelvin (Microbiology and Molecular Biology Reviews, 67(1): 16-37, 2003).

Although in some embodiments, the vector or construct is adapted to be transferred into a plant via Agrobacterium-mediated transformation, the present invention also contemplates any suitable modifications to the genetic construct which facilitate bacterial mediated insertion into a plant cell via bacteria other than Agrobacterium sp., for example, as described in Broothaerts et al. (Nature 433: 629-633, 2005).

The ninth aspect of the invention extends to all genetic constructs essentially as described herein, which include further nucleotide sequences intended for the maintenance and/or replication of the genetic construct in prokaryotes or eukaryotes and/or the integration of the genetic construct or a part thereof into the genome of a eukaryotic or prokaryotic cell.

Those skilled in the art will be aware of how to produce the constructs described herein and of the requirements for obtaining the expression thereof, when so desired, in a specific cell or cell-type under the conditions desired. In particular, it will be known to those skilled in the art that the genetic manipulations required to perform the present invention may require the propagation of a genetic construct described herein or a derivative thereof in a prokaryotic cell such as an E. coli cell or a plant cell or an animal cell. Exemplary methods for cloning nucleic acid molecules are described in Sambrook et al. (2000, supra)

In a tenth aspect, the present invention provides a cell comprising the genetic construct of the ninth aspect of the invention.

The genetic construct may be introduced into a cell via any means known in the art, including those hereinbefore described.

The construct referred to above may be maintained in the cell as a DNA molecule, as part of an episome (eg. a plasmid, cosmid, artificial chromosome or the like) or it may be integrated into the genomic DNA of the cell.

As used herein, the term “genomic DNA” should be understood in its broadest context to include any and all DNA that makes up the genetic complement of a cell. As such, the genomic DNA of a cell should be understood to include chromosomes, mitochondrial DNA, plastid DNA, chloroplast DNA, endogenous plasmid DNA and the like. As such, the term “genomically integrated” contemplates chromosomal integration, mitochondrial DNA integration, plastid DNA integration, chloroplast DNA integration, endogenous plasmid integration, and the like.

The cell may be any prokaryotic or eukaryotic cell. As such, the cell may be a prokaryotic cell such as a bacterial cell including an E. coli cell or an Agrobacterium spp. cell, or an archaea cell. The cell may also be a eukaryotic cell including a fungal cell such as a yeast cell or mycelial fungus cell; an animal cell such as a mammalian cell or an insect cell; or a plant cell. In some embodiments the cell is a plant cell. In some embodiments, the plant cell is a monocot plant cell, a Poales plant cell, or a cereal crop plant cell.

In an eleventh aspect, the present invention provides a multicellular structure, as hereinbefore defined, comprising one or more of the cells of the tenth aspect of the invention.

As mentioned above, in some embodiments, the cell is a plant cell and as such, the present invention should be understood to specifically include a whole plant, plant tissue, plant organ, plant part, plant reproductive material, or cultured plant tissue, comprising one or more cells of the eleventh aspect of the invention.

In some embodiments, the present invention provides a monocot plant, a Poales plant or a cereal crop plant or part thereof, comprising one or more cells of the tenth aspect of the invention.

In some embodiments, the present invention provides cereal grain comprising one or more cells of the tenth aspect of the invention.

In a twelfth aspect, the present invention provides a method for predicting the level of xylan production in an organism, the method comprising:
determining the expression level of a XynS nucleic acid sequence and/or a XynS polypeptide in one or more cells of the organism;
wherein the expression level of a XynS nucleic acid sequence and/or a XynS polypeptide is positively correlated with xylan production in the organism;
predicting the level of xylan production in the organism on the basis of the expression level of a XynS nucleic acid sequence and/or a XynS polypeptide in one or more cells of the organism.

In some embodiments of the twelfth aspect of the invention, the XynS xylan synthase comprises an amino acid sequence having at least 50% identity to one or more of: GenBank accession number AAR29963; GenBank accession number AAR29965; SEQ ID NO: 1, SEQ ID NO: 3 and/or SEQ ID NO: 5.

In some embodiments of the twelfth aspect of the invention the XynS nucleic acid comprises one or more of:
a nucleotide sequence which comprises at least 50% nucleotide sequence identity to one or more of: the nucleotide sequence set forth in GenBank accession number AY483151, GenBank accession number AY483153, SEQ ID NO: 2, SEQ ID NO: 4 and/or SEQ ID NO: 6; and/or
a nucleotide sequence which hybridises to a nucleic acid molecule comprising the nucleotide sequence set forth in one or more of: GenBank accession number AY483151, GenBank accession number AY483153, SEQ ID NO: 2, SEQ ID NO: 4 and/or SEQ ID NO: 6 under stringent conditions.

Methods for determining the level and/or pattern of expression of a nucleic acid or polypeptide are known in the art.

Exemplary methods of the detection of RNA expression include methods such as quantitative or semi-quantitative reverse-transcriptase PCR (eg. see Burton et al, Plant Physiology 134: 224-236, 2004), in-situ hybridization (eg. see Linnestad et al, Plant Physiology 118: 1169-1180, 1998); northern blotting (eg. see Mizuno et al, Plant Physiology 132: 1989-1997, 2003); and the like.

Exemplary methods for the expression of a polypeptide include Western blotting (eg. see Fido et al, Methods Mol Biol. 49: 423-37, 1995); ELISA (eg. see Gendloff et al, Plant Molecular Biology 14: 575-583); immunomicroscopy (eg. see Asghar et al, Protoplasma 177: 87-94, 1994) and the like.

In some embodiments, the expression level of the expression level of a XynS nucleic acid sequence and/or a XynS polypeptide may be determined by determining the copy number of XynS nucleic acids present in the genomic DNA of one or more cells of the organism. In these embodiments the copy number of a XynS nucleic acid in the genome of the cell is positively correlated with the expression level of the expression level of a XynS nucleic acid sequence and/or a XynS polypeptide. In some embodiments, the presence of at least 2, at least 3, at least 4, at least 5, at least 6, at least 7, at least 8, at least 9, at least 10, at least 20, at least 30, at least 40 or at least 50 copies of a XynS nucleic acid in the genome of one or more cells of the organism is associated with relatively high xylan production in the organism.

In some embodiments, the method of the twelfth aspect of the invention is adapted to predicting the level of xylan production in a plant. In some embodiments, the plant may be any of: a monocot plant, a cereal crop plant, a wheat plant, a barley plant and/or a rice plant.

In some embodiments, the method of the twelfth aspect of the invention may be used to predict the level of xylan production in an organism and then select individual organisms on the basis of the predicted level of xylan production. For example, in the case of plants, plants having a desired level of xylan production may be selected for cultivation and/or may be selected for breeding programs to produce plant lines having a desired level of xylan production.

The present invention is further described with reference to the following non-limiting examples:

### BRIEF DESCRIPTION OF THE FIGURES

Certain embodiments are illustrated by the following figures. It is to be understood that the following description is for the purpose of describing particular embodiments only and/or for describing comparative examples, and is not intended to be limiting with respect to the description.

FIG. 1 shows structures of heteroxylans from higher plants. A. Comparison of the chemical structures and conformations of (1,4)-β-glucan (cellulose, upper structure) and (1,4)-β-xylan (lower structure). Overall the shapes and dimensions of these polysaccharides are similar, with the major difference the absence of the hydroxymethyl group (—CH₂OH) on C(O)5 of the (1,4)-β-xylan. B. Diagrammatical representation of heteroxylan structures from higher plants, showing the (1,4)-β-xylan backbone with various monosaccharide substituents. C. Diagrammatical representation of the arabinoxylan from the mucilage of Plantago ovata, where the single xylosyl and trisaccharide substituents are apparent.

FIG. 2 shows transcript abundance in developing barley endosperm. A. Transcript profiles for the HvCesA genes, showing the relative abundance of the HvCesA3 mRNA (GenBank accession number AY483151.1) compared with the other members of the HvCesA gene family. B. Similar developmental profiles for the HvCslF (1,3; 1,4)-β-glucan synthase gene transcripts in developing barley endosperm, where it can be seen that the HvCslF6 gene transcripts are most abundant and are present at similar levels as those of the HvCesA3 gene.

FIG. 3 shows developing fruits of Plantago ovata. A. During the first 12 days post anthesis, the seed increased in size to about 5 mm. The integument layer was dissected out 0-6 days after anthesis, when the fruit was 1 mm or less in diameter. B. The seed of Plantago ovata at the 3 mm stage, showing remnants of the integument adhering to the seed. The transverse section links the seed surface structure with a diagrammatic representation of a seed section.

FIG. 4 shows immunocytochemistry of cell walls in transgenic barley lines. Sections of the grain were probed with the LM11 antibody, which is specific for arabinoxylans. Immuno-gold labeling shows the increased level of gold binding in cell walls of the transgenic lines compared with controls. Panel A shows HvCesA3 transformed barley. Panel C shows HvCesA5/7 transformed barley. Panels B and D show the respective empty-vector controls.

FIG. 5 shows a phylogenetic tree of cellulose synthase and cellulose synthase-like gene families in higher plants (from Fincher, Plant Physiol 149, 27-37, 2009). Cellulose synthase (CesA) and cellulose synthase-like (Csl) families from plants contain about 50 genes.

FIG. 6 shows total arabinoxylan percentage (w/w) in rice calli transformed with Plantago ovata XynS genes PoC321, PoC217 and PoC420 and empty-vector (pMDC32) controls, wherein A and B represent replicate samples.

FIG. 7 shows in situ hybridisation of PoC217 (C) and PoC321 (E) with antisense probes and the PoC217 sense control (D) and no probe (NP) in developing Plantago ovata integuments. ov—ovule, ca—carpel, pl—placenta, ie—integument epithelium.

FIG. 8 shows the grain arabinoxylan (AX) content in barley transformed with XynS expression constructs AsGLO:CesA3, AsGLO:CesA5/7, 35S:CesA3 and 35S:CesA5/7.

FIG. 9 shows immunocytochemistry in transgenic rice callus transformed with CaMV35:PoC321 and CaMV35S:PoC217. Panel A shows transgenic empty vector control probed with the LM11 antibody. Panel B shows CaMV35S:PoC321-transformed callus probed with the LM11 antibody. Panel C shows CaMV35S:PoC217-transformed callus probed with the LM11 antibody.

FIG. 10 shows the grain arabinoxylan (AX) content in rice grain transformed with XynS expression constructs AsGLO:PoC217, AsGLO:PoC321 and AsGLO:PoC420.

FIG. 11 is a table showing polysachharide composition as determined by methylation analysis of linkage positions in the destarched alcohol insoluble residue (‘AIR’) of whole barley grain. The controls include barley cv. Golden promise parent plant material (GP) and two empty vector (EV) transgenic lines. The four transgenic lines overexpress CesA3 (XynS) and CesA5/7 (XynS) under the control the 35S and AsGLO promoters.

### EXAMPLE 1

### CesA Transcript Profiling in Barley

In the mature barley grain, isolated cell walls from the starchy endosperm contain only about 3% w/w cellulose; the major polysaccharides in these walls are (1,3; 1,4)-β-glucans (about 70% w/w) and arabinoxylans (about 20% w/w).

The initial clues that the CesA Glade might also harbour genes involved in (1,4)-β-xylan biosynthesis emerged during transcript profiling of the endosperm of developing barley grain, where unexpectedly high levels of CesA gene transcripts were detected.

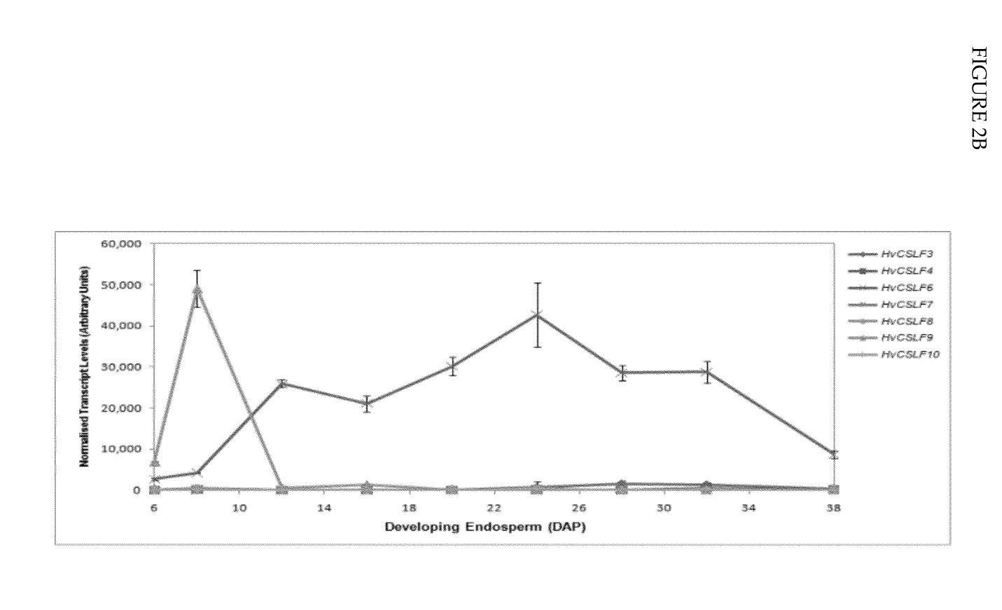

As shown in FIG. 2, levels of HvCesA3 (GenBank accession AY483151.1) transcripts in the developing barley endosperm were similar to those of the HvCslF6 gene (Genbank accession EU267181.1), which is known to be the major gene involved in synthesis of the much more abundant (1,3; 1,4)-β-glucan in the grain.

The orthologous gene of HvCesA3 in maize is ZmCesA5, which is the most highly expressed CesA gene in developing maize endosperm (Appenzeller et al., Cellulose 11(3-4): 287, 2004).

The levels of HvCesA2 (Genbank accession AY483152.1) transcripts were also reasonably high in the developing barley endosperm, compared with the very low levels of the other HvCesA genes (FIG. 2).

### EXAMPLE 2

### CesA Transcript Profiling in Plantago ovata

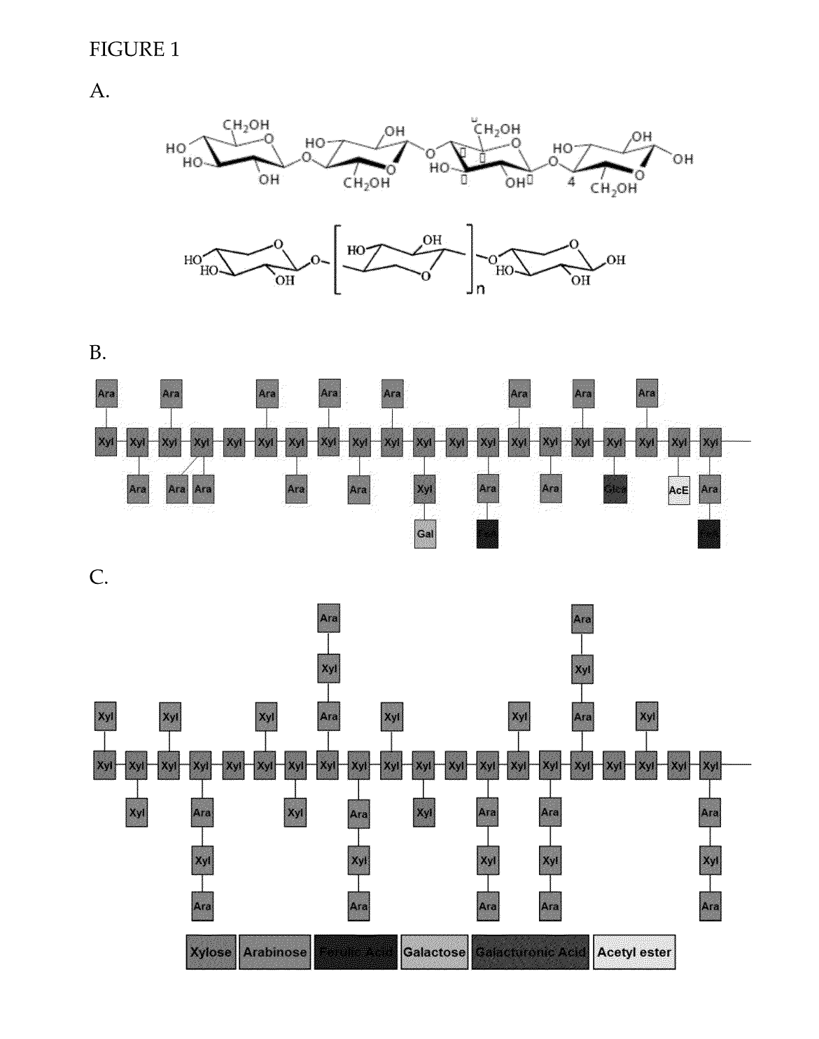

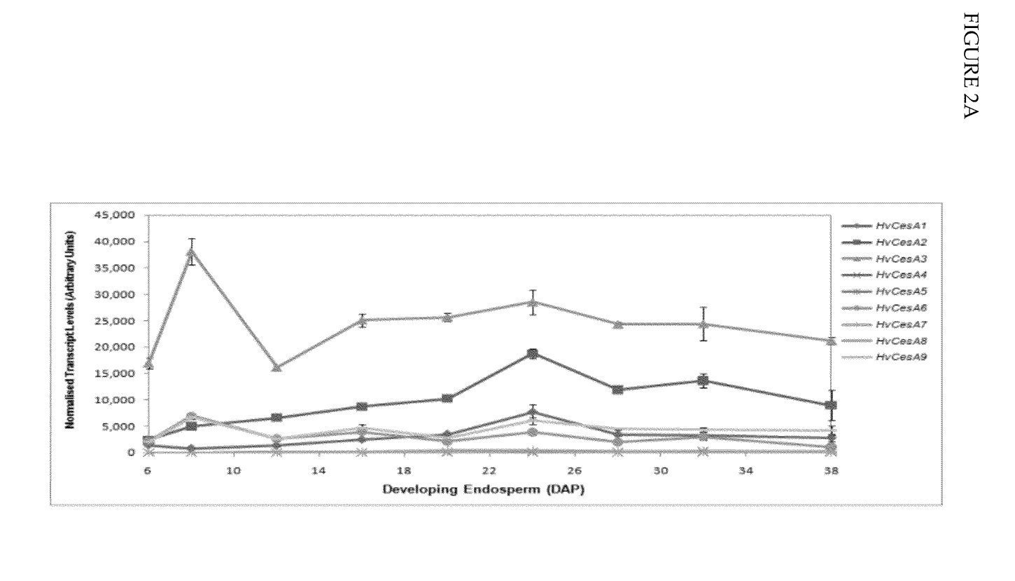

To further investigate the possibility that CesA genes might be involved in xylan synthesis in higher plants, transcript profiles were generated from the integuments of seeds from Plantago ovata, which, upon wetting of the seed, excrete mucilage that is composed predominantly of heteroxylan and essentially no cellulose. The heteroxylan component of the mucilage is unusual, insofar as the (1,4)-β-xylan backbone is heavily substituted with single xylopyranosyl residues at position C(O)₃ and with the trisaccharide L-Araf-α-(1,3)-D-Xylp-β-(1,3)-L-Araf (see FIG. 1C).

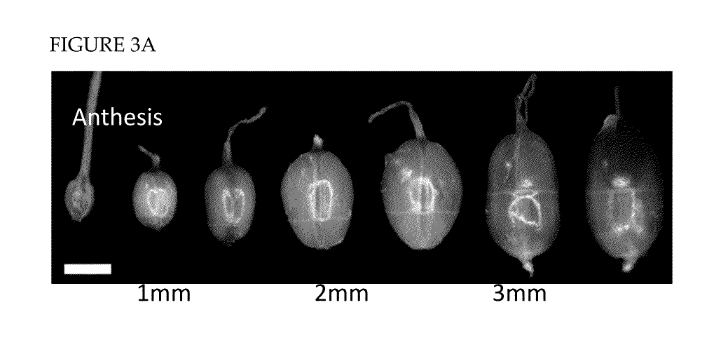

The integument layer of cells was dissected from developing fruit of Plantago ovata 0-6 days after anthesis, when the fruit was 1 mm or less in diameter (see FIG. 3). Extracted RNA was subjected to deep sequencing, which generated about 15 million reads and more than 37,000 contigs. Selected genes within the 200 most abundant RNA sequences are shown in Table 2, below.

TABLE 2
Relative abundance of selected gene transcripts in the integument
from Plantago ovata seeds 3-4 days post anthesis. The selected
genes are those that have clear connections with cell wall
biosynthesis, re-modelling or degradation, or with the synthesis
of sugar-nucleotides that are known to act as sugar donors
during cell wall biosynthesis in plants.

| Ranking | | Length of contig | |
|---|---|---|---|
| (out of 37,000) | Gene | (bp) | Reads |
| 8 | CesA | 3811 | 27481 |
| 9 | GT61 | 1862 | 26645 |
| 15 | GT61 | 1619 | 23387 |
| 35 | UXS | 1419 | 18938 |
| 41 | GT61 | 1667 | 17689 |
| 42 | GT47 | 2273 | 17574 |
| 43 | CesA | 3723 | 17409 |
| 49 | GT31 | 2517 | 16754 |
| 51 | CesA | 3549 | 16437 |
| 59 | UDP-GluA epimerase | 1720 | 15384 |
| 64 | GT61 | 1031 | 14943 |
| 68 | Cellulase (GH9) | 2372 | 14743 |
| 74 | UXS | 1158 | 14059 |
| 79 | Cellulase (GH9) | 2518 | 13610 |
| 83 | UDP-GluA epimerase | 1893 | 13285 |
| 90 | Cellulase (GH9) | 1715 | 12980 |
| 105 | Cellulase (GH9) | 2301 | 11889 |
| 106 | UXS | 1792 | 11837 |
| 121 | UDP-Ara mutase (GT75) | 1481 | 10712 |
| 149 | Xylanase | 2993 | 9313 |
| 151 | Cobra-like | 3443 | 9225 |
| 166 | GT61 | 924 | 8827 |
| 167 | DUF579 | 1258 | 8813 |
| 172 | GT47 | 2045 | 8619 |
| 179 | GT61 | 905 | 8493 |
| 187 | GT8 | 2134 | 8149 |

The RNA-Seq data showed that three PoCesA (XynS) genes were amongst the top 51 most abundant transcripts in the developing Plantago ovata integuments (Table 2). These genes were designated PoC321 (SEQ ID NO: 2), PoC217 (SEQ ID NO: 4) and PoC420 (SEQ ID NO: 6).

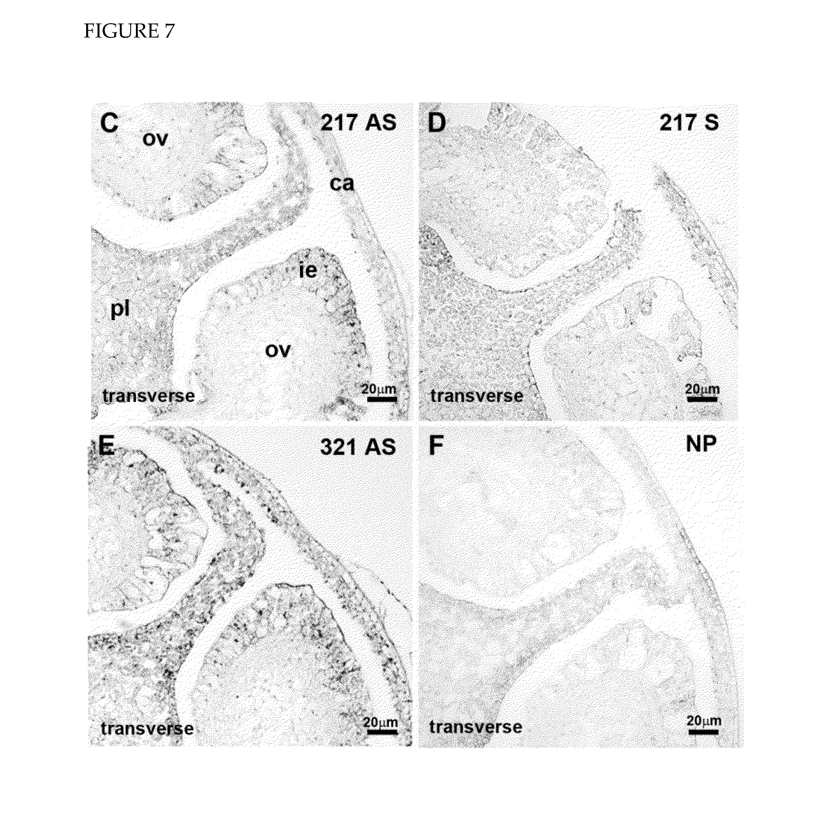

Although it is well known that this layer produces the heteroxylan mucilage, when this section was probed with the LM10 and LM11 antibodies, only weak binding was observed. This is presumably attributable to the difference in structure of the Plantago ovata heteroxylan (FIG. 1C) compared with the more common structure (FIG. 1B) of heteroxylans used to raise the antibodies (Knox, Curr. Opin. Plant Biol. 11: 308-313, 2004). However, in situ hybridization with two of the PoCesA probes showed that mRNA corresponding to these genes was abundant in the outer layers of the collapsing, liquefying integument (FIG. 7).

### EXAMPLE 3

### HvCesA3 (XynS) and HvCesA5/7 (XynS) Transformation into Barley

In order to determine whether over-expression of the gene led to increased levels of arabinoxylan in grain, barley (cv. Golden Promise) was transformed with the HvCesA3 (XynS—GenBank accession AY483151) and HvCesA5/7 (XynS—GenBank accession AY483153) genes under the control of the CaMV35S and AsGLO promoters as described in Burton et al. (Plant Biotechnology 9: 117-135, 2011)

Grains from the first generation of the XynS transgenic lines (T₁) were analysed for arabinoxylan content, which was hydrolysed prior to separation and quantitation of constituent monosaccharides by HPLC. Selected T₁ lines with higher or lower arabinoxylan levels in their grain were taken to the T₂ and T₃ generations, where segregation of the transgene was largely complete.

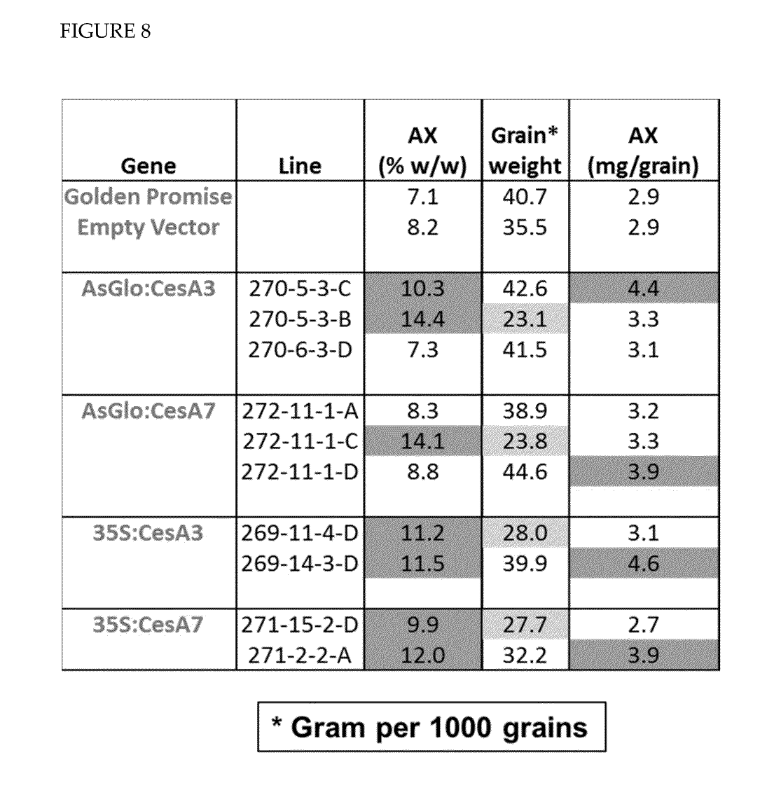

As shown in FIG. 8, transgenic lines showed either increases in arabinoxylan content or no change, when compared with the wild type barley and empty vector controls.

The highest arabinoxylan value obtained was 14.4% (w/w) for one line. When the arabinoxylan content was expressed on a per grain basis, two lines with >4 mg/grain (4.4 and 4.6) arabinoxylan were obtained, which represented an approximately 50% increase compared with the control lines.

### EXAMPLE 4

### Histology of Barley HvCesA3 (XynS) and HvCesA5/7 (XynS) Transformants

To further examine these apparent increases in arabinoxylan content in the walls of endosperm cells of the AsGLO:HvCesA3 (XynS) and AsGLO:HvCesA5/7 (XynS) transgenic lines, sections of the grain were fixed and probed with the LM11 antibody, which recognizes arabinoxylan (Knox, Curr Opin Plant Biol 11: 308-313, 2008).

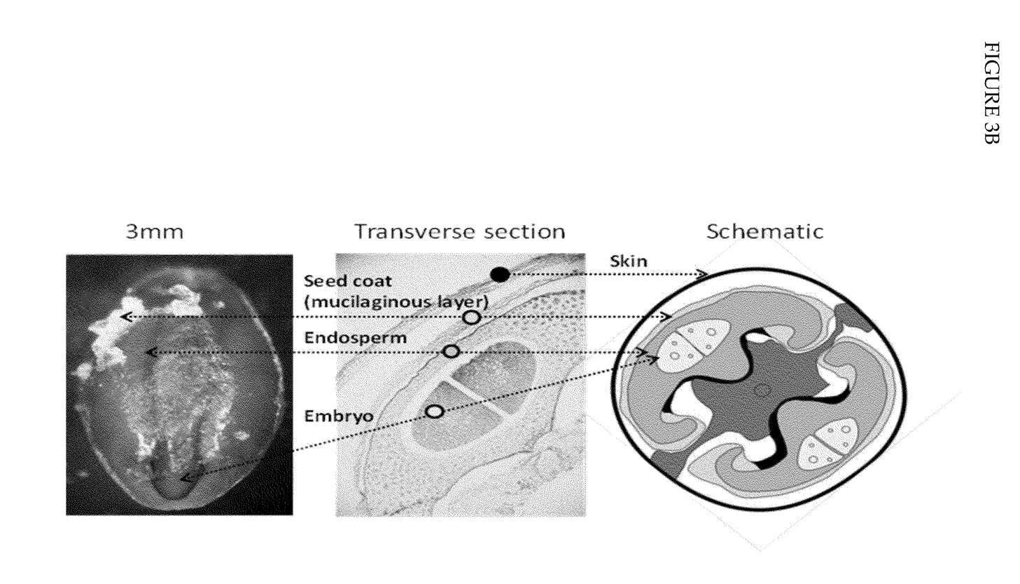

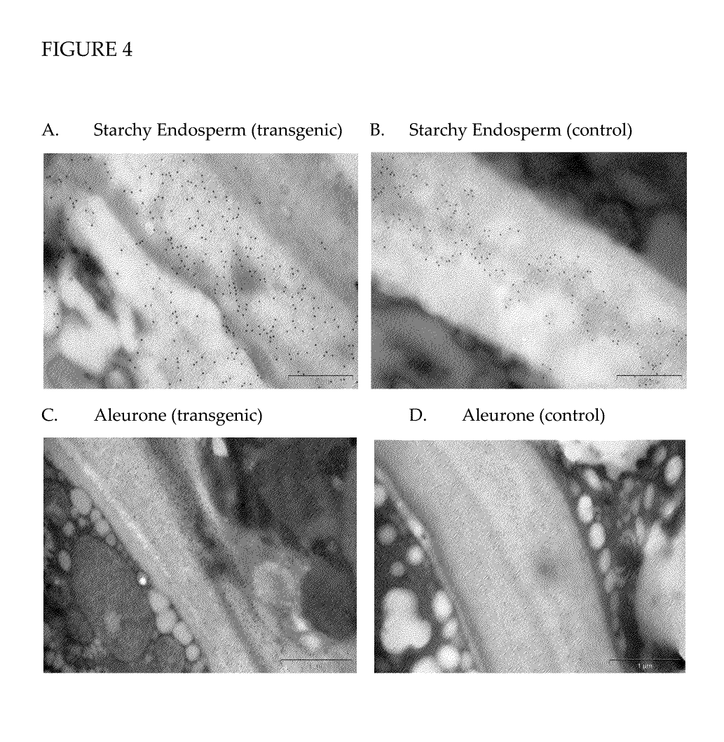

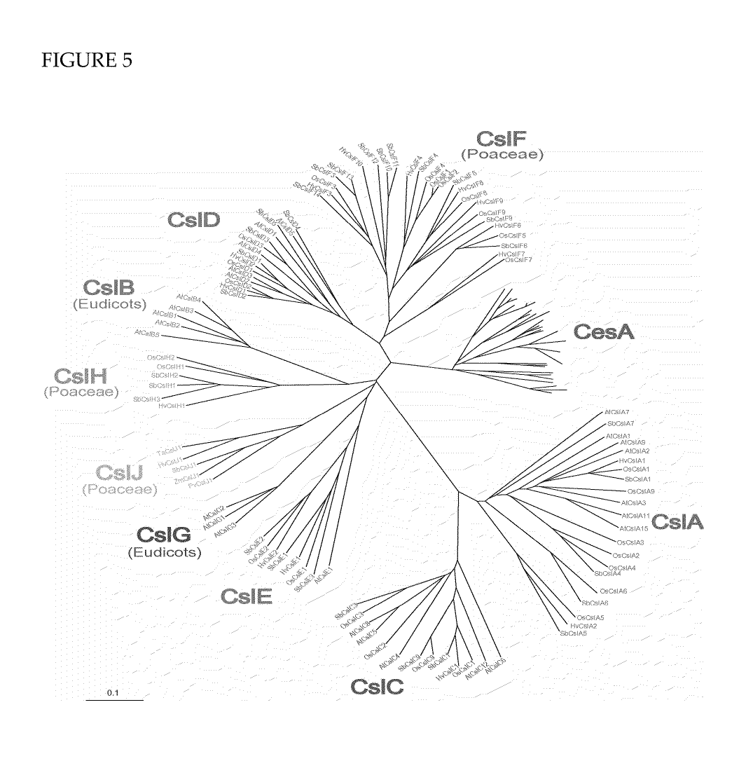

As shown in FIG. 4, in the cell walls of the starchy endosperm of transgenic grain in both transgenic lines noticeably higher levels of antibody binding was evident compared with the empty vector controls.

In some cases, ectopic deposition of large bodies of non-wall, antibody-positive material was detected in the cytoplasm and in the extracellular space. We conclude that the ectopic deposition of the arabinoxylan in transgenic lines probably results from overloading of the cellular mechanisms that mediate the synthesis and deposition of polysaccharides into the wall.

The antibody data were consistent with the compositional analyses, where antibody binding in the starchy endosperm of transgenic lines (FIG. 4A), which contained 14.4% arabinoxylan (w/w), was very much higher than in the control line (FIG. 4B), which contained 8.1% (w/w) arabinoxylan.

It is also noteworthy that 35S:HvCesA4 and 35S:HvCesA8 transgenic barley lines show no additional arabinoxylan in the grain.

### EXAMPLE 5

### Transformation of Plantago ovata XynS Nucleotide Sequences into Rice (Oryza sativa)

Full length cDNAs of three Plantago ovata XynS nucleotide sequences, PoC217 (SEQ ID NO: 4), PoC321 (SEQ ID NO: 2) and PoC420 (SEQ ID NO: 6), were amplified and cloned into the entry vector pCR8/TOPO/TA. Each of the inserts were transferred via an LR Gateway reaction into the binary vector pMDC32 (Curtis and Grossniklaus, Plant Physiol. 133(2): 462-469, 2003) which carries the CaMV35S promoter or pRB474 which carries the AsGLO promoter. Each construct was then used to transform the Agrobacterium strain AGL1.

Rice callus cultures were transformed using Agrobacterium-mediated transformation as described in Nishimura et al. (Nature Protocols 1: 2796-2802, 2007) to produce transgenic callus material. This was either control material carrying the empty vector (Rice control) or material transgenic for any of PoC217, PoC321 or PoC420.

Transgenic callus material from the control and PoC constructs was collected and freeze dried. The material was ground and acid hydrolysed to break down polysaccharides into constituent sugars. An aliquot of the liquid phase of the treatment was run on a high performance liquid chromatograph (HPLC) to determine the relative amounts of the monosaccharides present.

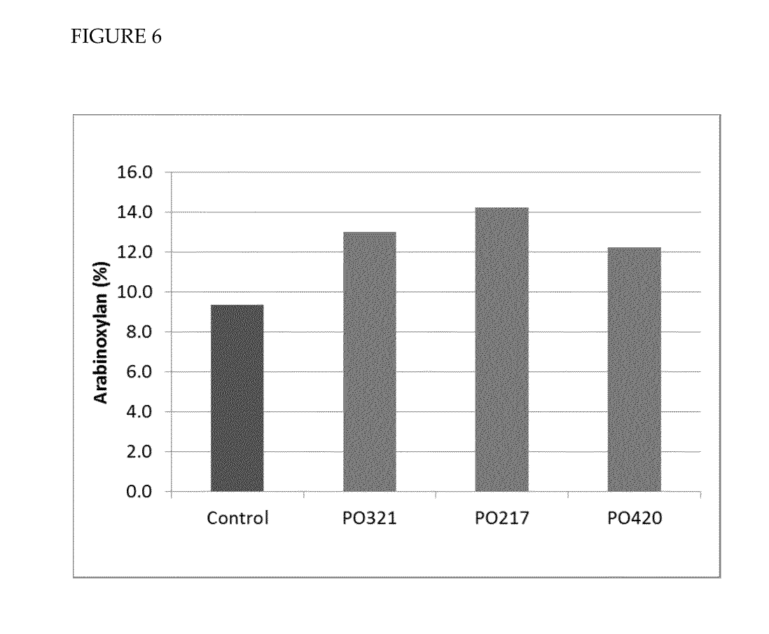

FIG. 6 shows the arabinoxylan content of rice calli transformed with the CaMV35S:PoC217 (XynS), CaMV35S:PoC321 (XynS) and CaMV35S:PoC420 (XynS) constructs. The total amount of arabinoxylan was increased from a control level of 6.8% to −8.8% in the PoC transgenic lines.

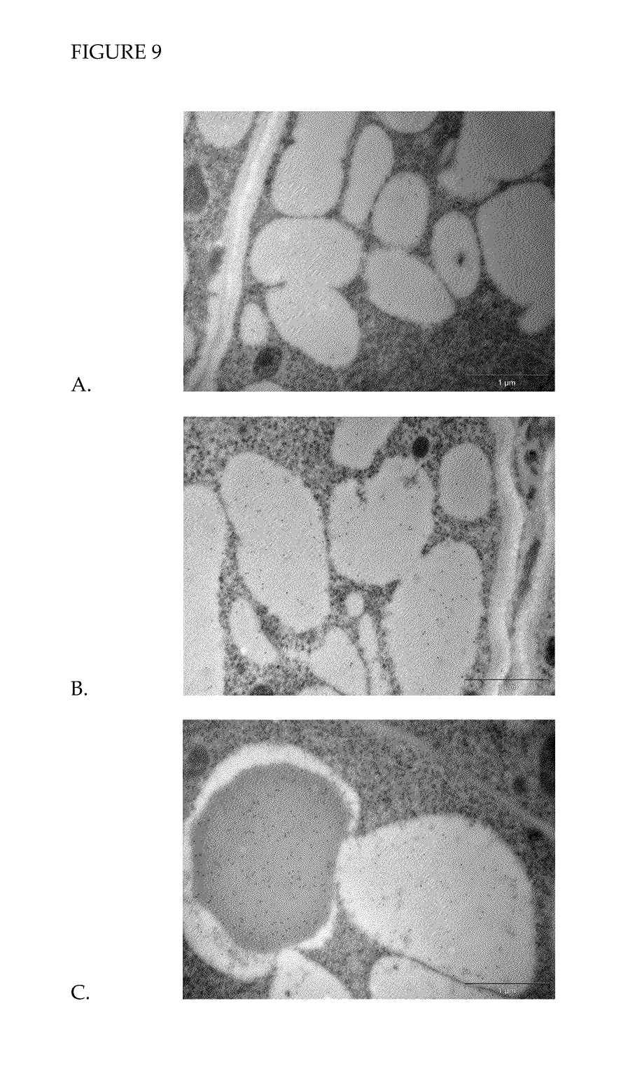

To further examine the increase in arabinoxylan content in CaMV35S:PoC321 (XynS) and CaMV35S:PoC217 (XynS) transgenic rice calli, sections of the calli were fixed and probed with the LM11 antibody, which recognizes arabinoxylans (Knox, 2008, supra). As shown in FIG. 9 noticeably higher levels of antibody binding was evident, in the transgenic calli compared with the empty vector controls.

### EXAMPLE 6

### Analysis of Arabinoxylan Content in AsGLO:PoC217 (XynS), AsGLO:PoC321 (XynS) and AsGLO:PoC420 (XynS) Transformed Rice Grain

Rice calli transformed with each of AsGLO:PoC217 (XynS), AsGLO:PoC321 (XynS) and AsGLO:PoC420 (XynS) as described in Examples 5 were regenerated into plants and grain from the first generation of the transgenic lines (T₁) was analysed for arabinoxylan content, which was hydrolysed prior to separation and quantitation of constituent monosaccharides by HPLC.

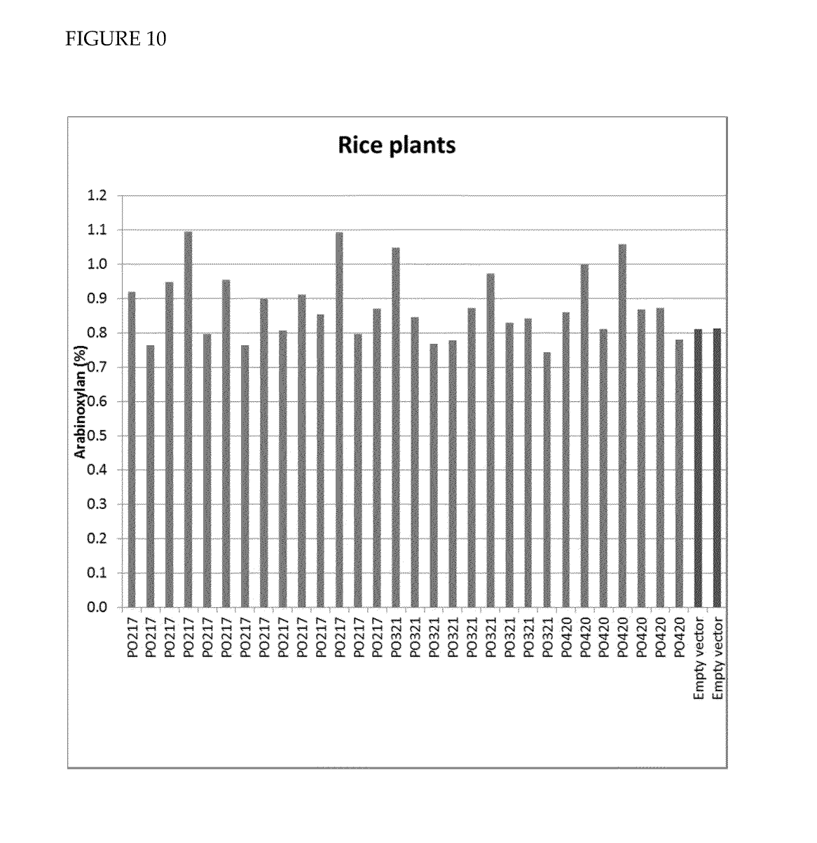

As shown in FIG. 10, several transgenic lines showed increases in arabinoxylan content when compared with the empty vector controls.

### EXAMPLE 7

### Polysaccharide Composition Analysis of XynS-Overexpressing Barley Grain

The polysaccharide composition of XynS overexpressing barley grain was determined by methylation analysis of linkage positions in the destarched alcohol insoluble residue (‘AIR’) of whole barley grain using a method as described by Pettolino et al. (Nature Protocols 7: 1590-1607, 2012)

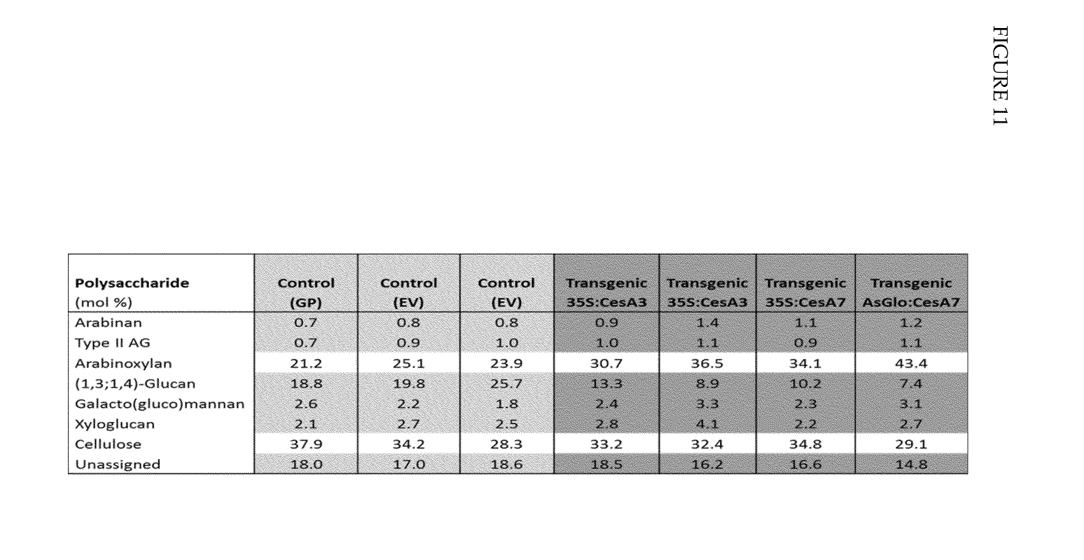

As shown in FIG. 11 the major polysaccharides in the grain are cellulose, mixed-linkage beta glucan and heteroxylan. There is no significant changes in level of cellulose between the controls and the XynS overexpressing grain. However, there is a substantial increase (˜50%) in arabinoxylan observed in the XynS overexpressing grain relative to the controls.

### EXAMPLE 8

### Model for Possible Interaction with XynS and Other Biosynthetic Enzymes in Xylan Biosynthesis

Presented below are hypothetical models for how XynS might interact with other biosynthetic enzymes in the biosynthesis of xylan. Notwithstanding, the present invention should not be considered in any way limited to the mode of action described below.

Heteroxylan biosynthesis in higher plants may involve XynS working in conjunction with other proteins such as GT47 and GT61 proteins.

For example, it is possible that these proteins form a single multi-enzyme complex in which the XynS and enzymes such as GT47 proteins are jointly involved in the synthesis of the (1,4)-β-xylan backbone, while various enzymes such as GT61 enzymes add the arabinosyl and xylosyl substituents to the main chain.

An alternative to the single multi-enzyme complex model involves a two-phase assembly of the polysaccharide, as suggested previously for (1,3; 1,4)-β-glucan synthesis in species from the grass family (eg. see Doblin et al., Proc. Natl. Acad. Sci. USA 106: 5996-6001, 2009; Fincher, Plant Physiol. 149: 27-37, 2009; Burton et al., Nature Chem. Biol. 6: 724-732, 2010). In the case of (1,3; 1,4)-β-glucan synthesis, it was proposed that in phase I oligosaccharides are synthesised in the Golgi apparatus by the CslF protein, transported to the plasma membrane via a membrane-bound carrier molecule and, in the second phase, the oligosaccharides are polymerized into the final (1,3; 1,4)-β-glucan polysaccharide at the plasma membrane or in the periplasmic space. Although the transferase enzyme that would be required for the final polymerization of the oligosaccharides has not been identified, it has been suggested that GSL (1,3)-β-glucan synthases or xyloglucan endotransglycosylases (XET) could be possible candidates for the role.

It is possible that the same two-phase assembly process might also occur during heteroxylan synthesis. For example, (1,4)-β-oligoxylosides may be synthesized by, for example, GT47 enzymes, probably in the Golgi, where arabinosyl and xylosyl substituents are also added to the (1,4)-β-oligoxylosides through the action of, for example, GT61 enzymes. The (1,4)-p-oligoxylosides could subsequently be polymerized into the final (1,4)-β-xylan by the XynS enzymes, possibly at the plasma membrane. Equally, the XynS enzymes might be involved in (1,4)-β-oligoxyloside synthesis in the Golgi, while enzymes such as GT47 are responsible for the polymerization of the oligosaccharides at the plasma membrane.

Reference is made to standard textbooks of molecular biology that contain methods for carrying out basic techniques encompassed by the present invention, including DNA restriction and ligation for the generation of the various genetic constructs described herein. See, for example, Maniatis et al, Molecular Cloning: A Laboratory Manual (Cold Spring Harbor Laboratory Press, New York, 1982) and Sambrook et al. (2000, supra).

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Reference to any prior art in this specification is not, and should not be taken as, an acknowledgment or any form of suggestion that this prior art forms part of the common general knowledge in any country.

Also, it must be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The subject headings used herein are included only for the ease of reference of the reader and should not be used to limit the subject matter found throughout the disclosure or the claims. The subject headings should not be used in construing the scope of the claims or the claim limitations.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

1. A method for modulating the level of xylan produced by a cell, the method comprising modulating the level and/or activity of a XynS xylan synthase in the cell.

2. A method for modulating the level and/or activity of a xylan synthase in a cell, the method comprising modulating the expression of a XynS nucleic acid in the cell.

3. A method for producing xylan, the method comprising transforming a cell with an isolated XynS nucleic acid and allowing the cell to express the isolated XynS nucleic acid.

4. The method of any one of claims 1 to 3 wherein the cell is a plant cell.

5. The method of claim 4 wherein the cell is a monocot plant cell.

6. The method of claim 4 wherein the cell is a cereal crop plant cell.

7. The method of any one of claims 1 to 6 wherein the XynS xylan synthase comprises an amino acid sequence having at least 50% identity to one or more of: GenBank accession number AAR29963; GenBank accession number AAR29965; SEQ ID NO: 1, SEQ ID NO: 3 and/or SEQ ID NO: 5.

8. The method of any one of claims 2 to 7 wherein the XynS nucleic acid comprises:
a nucleotide sequence which comprises at least 50% nucleotide sequence identity to one or more of: the nucleotide sequence set forth in GenBank accession number AY483151, GenBank accession number AY483153, SEQ ID NO: 2, SEQ ID NO: 4 and/or SEQ ID NO: 6; and/or
a nucleotide sequence which hybridises to a nucleic acid molecule comprising the nucleotide sequence set forth in one or more of: GenBank accession number AY483151, GenBank accession number. AY483153, SEQ ID NO: 2, SEQ ID NO: 4 and/or SEQ ID NO: 6 under stringent conditions.

9. Xylan produced according to the method of any one of claims 3 to 8.

10. A cell comprising:
a modulated level and/or activity of XynS xylan synthase relative to a wild type cell of the same taxon; and/or
modulated expression of a XynS nucleic acid relative to a wild type cell of the same taxon.

11. The cell of claim 10 wherein the cell further comprises a modulated level of xylan relative to a wild type cell of the same taxon.

12. The cell of claim 10 or 11 wherein the cell is produced according to the method of any one of claims 1 to 9.

13. The cell of any one of claims 10 to 12 wherein the cell is a plant cell.

14. The cell of claim 13 wherein the cell is a monocot plant cell.

15. The cell of claim 13 wherein the cell is a cereal crop plant cell.

16. The cell of any one of claims 10 to 15 wherein the XynS xylan synthase comprises an amino acid sequence having at least 50% identity to one or more of: GenBank accession number AAR29963; GenBank accession number AAR29965; SEQ ID NO: 1, SEQ ID NO: 3 and/or SEQ ID NO: 5.

17. The cell of any one of claims 10 to 16 wherein the XynS nucleic acid comprises:
a nucleotide sequence which comprises at least 50% nucleotide sequence identity to one or more of: the nucleotide sequence set forth in GenBank accession number AY483151, GenBank accession number AY483153, SEQ ID NO: 2, SEQ ID NO: 4 and/or SEQ ID NO: 6; and/or
a nucleotide sequence which hybridises to a nucleic acid molecule comprising the nucleotide sequence set forth in one or more of: GenBank accession number AY483151, GenBank accession number AY483153, SEQ ID NO: 2, SEQ ID NO: 4 and/or SEQ ID NO: 6 under stringent conditions.

18. A multicellular structure comprising one or more cells according to any one of claims 10 to 17.

19. The multicellular structure of claim 18 wherein the multicellular structure is selected from a whole plant, a plant tissue, a plant organ, a plant part, plant reproductive material and/or cultured plant tissue.

20. The multicellular structure of claim 18 or 19 wherein the multicellular structure comprises a cereal crop plant or a tissue, organ or part thereof.

21. The multicellular structure of claim 20 wherein the multicellular structure comprises a cereal grain.

22. A cereal grain comprising a modulated level of xylan, wherein the grain comprises one or more cells comprising:
a modulated level and/or activity of a XynS xylan synthase; and/or
modulated expression of a XynS nucleic acid.

23. The cereal grain of claim 22 wherein the XynS xylan synthase comprises an amino acid sequence having at least 50% identity to one or more of: GenBank accession number AAR29963; GenBank accession number AAR29965; SEQ ID NO: 1, SEQ ID NO: 3 and/or SEQ ID NO: 5.

24. The cereal grain of claim 22 or 23 wherein the XynS nucleic acid comprises:
a nucleotide sequence which comprises at least 50% nucleotide sequence identity to one or more of: the nucleotide sequence set forth in GenBank accession number AY483151, GenBank accession number AY483153, SEQ ID NO: 2, SEQ ID NO: 4 and/or SEQ ID NO: 6; and/or
a nucleotide sequence which hybridises to a nucleic acid molecule comprising the nucleotide sequence set forth in one or more of: GenBank accession number AY483151, GenBank accession number AY483153, SEQ ID NO: 2, SEQ ID NO: 4 and/or SEQ ID NO: 6 under stringent conditions.

25. Flour comprising flour produced by the milling of the grain of any one of claims 22 to 24.

26. A genetic construct or vector comprising an isolated XynS nucleic acid or a complement, reverse complement or fragment thereof.

27. The genetic construct or vector of claim 26 wherein the XynS nucleic acid encodes a XynS xylan synthase comprising an amino acid sequence having at least 50% identity to one or more of: GenBank accession number AAR29963; GenBank accession number AAR29965; SEQ ID NO: 1, SEQ ID NO: 3 and/or SEQ ID NO: 5.

28. The genetic construct or vector of claim 26 or 27 wherein the XynS nucleic acid comprises one or more of:
a nucleotide sequence which comprises at least 50% nucleotide sequence identity to one or more of: the nucleotide sequence set forth in GenBank accession number AY483151, GenBank accession number AY483153, SEQ ID NO: 2, SEQ ID NO: 4 and/or SEQ ID NO: 6; and/or
a nucleotide sequence which hybridises to a nucleic acid molecule comprising the nucleotide sequence set forth in one or more of: GenBank accession number AY483151, GenBank accession number AY483153, SEQ ID NO: 2, SEQ ID NO: 4 and/or SEQ ID NO: 6 under stringent conditions.

29. A cell comprising the genetic construct of any one of claims 26 to 28.

30. The cell of claim 29 wherein the cell is a plant cell.

31. The cell of claim 29 wherein the cell is a monocot plant cell.

32. The cell of claim 29 wherein the cell is a cereal crop plant cell.

33. A multicellular structure which comprises one or more of cells of any one of claims 29 to 32.

34. The multicellular structure of claim 33 wherein the multicellular structure is selected from a whole plant, a plant tissue, a plant organ, a plant part, plant reproductive material and/or cultured plant tissue.

35. The multicellular structure of claim 34 wherein the multicellular structure comprises a cereal crop plant or a tissue, organ or part thereof.

36. The multicellular structure of claim 35 wherein the multicellular structure comprises a cereal grain.

37. A method for predicting the level of xylan production in an organism, the method comprising:
determining the expression level of a XynS nucleic acid sequence and/or a XynS polypeptide in one or more cells of the organism;
wherein the expression level of a XynS nucleic acid sequence and/or a XynS polypeptide is positively correlated with xylan production in the organism;
predicting the level of xylan production in the organism on the basis of the expression level of a XynS nucleic acid sequence and/or a XynS polypeptide in one or more cells of the organism.

38. The method of claim 37 wherein the organism is a plant.

39. The method of claim 37 wherein the plant is a monocot plant.

40. The method of claim 37 wherein the plant is a cereal crop plant.

41. The method of any one of claims 37 to 40 wherein the XynS xylan synthase comprises an amino acid sequence having at least 50% identity to one or more of: GenBank accession number AAR29963; GenBank accession number AAR29965; SEQ ID NO: 1, SEQ ID NO: 3 and/or SEQ ID NO: 5.

42. The method of any one of claims 37 to 41 wherein the XynS nucleic acid comprises one or more of:
a nucleotide sequence which comprises at least 50% nucleotide sequence identity to one or more of: the nucleotide sequence set forth in GenBank accession number AY483151, GenBank accession number AY483153, SEQ ID NO: 2, SEQ ID NO: 4 and/or SEQ ID NO: 6; and/or
a nucleotide sequence which hybridises to a nucleic acid molecule comprising the nucleotide sequence set forth in one or more of: GenBank accession number AY483151, GenBank accession number AY483153, SEQ ID NO: 2, SEQ ID NO: 4 and/or SEQ ID NO: 6 under stringent conditions.


SEQUENCE LISTING (6 sequences)

SEQ ID NO: 1 - protein, 1053 aa, Plantago ovata
MAGLFAGSHGRNELHVLHTGTDDEHDRRSSIREQAAKICRICGDEIGLRENGDTFVACHECAFPVCRPCYEYERMEGQGTCPQCHTRYKRHKGCPKIEGDGEEENASDDFEDEFQLKNLQYDTHEPQHSENGEFKQQQNHVNVVATRSVAGSVTGKEFEAEKETYSSDEWKDRVEKWKTRQEKRGLVAKTDDGGNDNKDDDYEDEYLIAEARQPLWRKVPIASRLISPYRIVIVLRLIILCFFFHFRILTPAKDAYPLWIISVICEIWFGVSWILDQFPKWYPINRETYLDRLTLRFEREGEPSRLSQVDVYVSTVDPLKEPPIITANTVLSILAADYPVEKLSCYVSDDGASMLLFDSLAETAEFARRWVPFCKKYNVEPRAPEFYFSEKIDYLKDKVQPTFVKDRRAMKREYEEFKVRMNALVAKAQKKPEEGWVMQDGTPWPGNNTRDHPGMIQVYLGSEGALDVDGKELPRLVYVSREKRPGYQHHKKAGAMNALVRVSAVLTNAPFMLNLDCDHYLNNSKAIREAMCFLMDPQIGKKLCYVQFPQRFDGIDRHDRYANRNIVFFDINMKGLDGIQGPVYVGTGCVFNRQALYGYDPPASEKRAKMTCDCWPKWCCCCCGGSRKPKSKKKGLKALLGLGGLYSKKKKMMGKQYTRKPSGPVFDLEEIEEGLEGYDELEKSSLMSQKNFEKRFGMSPVFITSTLMENGGLPEGTSPGSLVKEAIHVISCGYEEKTEWGKEIGWIYGSITEDILTGFKMHCRGWRSVYCCPKRPAFKGSAPINLSDRLHQVLRWALGSVEIFLSRHCPLWYGYGGKLKYLERLAYINTIVYPFTSLALLAYCTLPAVCLLTGKFIVPTLNNLASIWFMALFLSIIATGVLELRWSGVSIEDWWRNEQFWVIGGLSAHLFAVFQGLLKVLAGVDTNFTVTSKAADDAEFGELYLFKWTTLLIPPTTIIILNMVGVVAGVSDAINNGYGSWGPLFGKLFFAFWVIVHLYPFLKGLMGKQNRTPTIVVLWSILLASIFSLVWVRIDPFLPKQTGPILKPCGVEC

SEQ ID NO: 2 - DNA, 3162 bp, Plantago ovata
atggctggcctctttgccggctctcacggccgcaatgaacttcatgttctccacacgggcactgatgacgagcatgacaggcgctcttcaatacgcgaacaagctgcgaaaatatgccggatttgtggagatgagatcggactaagggagaacggagatacattcgtggcgtgtcacgagtgtgcgtttccggtttgccgtccttgctacgaatacgaaaggatggagggtcaagggacctgccctcagtgtcacactcgttacaaacgccacaagggctgtcctaagattgaaggagatggcgaagaagaaaatgctagtgacgattttgaagatgagtttcagttgaagaatcttcagtatgacactcatgaacctcaacattcggaaaatggcgagttcaagcagcagcagaatcacgtcaatgtggtagctactcgttctgttgcaggaagtgttactggaaaggaatttgaagctgaaaaggaaacttacagcagcgatgagtggaaggatagagtggagaagtggaaaaccaggcaggagaagagaggtctcgttgccaaaacagacgatggaggaaatgacaacaaggatgatgattatgaagatgaatatcttattgctgaagctaggcagccactatggagaaaagttccgattgcttcccgcctcatcagcccttaccgcattgtcattgtccttaggttaatcatcctgtgtttcttcttccattttagaatcttgactccagcaaaggatgcttatcccttgtggattatctctgtgatatgtgagatatggtttggtgtatcctggattctggatcagttccctaagtggtatcctattaaccgagaaacctaccttgatcgcctcaccttgaggttcgagcgtgagggtgagcctagccggctttcccaggttgatgtctatgtgagtactgtggaccctctaaaggaacctcccattattaccgcaaacacagtcctttcaattttagcagctgactatccagtggagaaactgagttgctatgtttcggatgatggtgcctcgatgcttctctttgattcattggctgaaacagctgagttcgcgaggaggtgggtgcctttctgcaagaaatataacgtggagcctagggcacctgagttttatttctctgagaaaattgactacttgaaagataaggttcagcctacttttgtcaaggatcgcagagccatgaaaagagagtacgaagagtttaaggtaagaatgaatgcgttggtggccaaggctcagaagaaaccagaagaaggatgggttatgcaggacgggactccgtggcctggaaacaacactagggatcaccctggaatgattcaggtatatttgggaagtgaaggtgcactggatgttgatggtaaggagctgccacggctagtatatgtctctcgtgagaaacgaccaggctaccagcatcataagaaagcaggagcaatgaatgcactagtacgggtgtctgctgtgctaactaacgcaccattcatgttgaacttggattgtgatcattaccttaacaatagcaaggcaattagggaagctatgtgcttcttaatggacccccaaatcgggaaaaaactttgctatgttcaatttccacagagatttgatggtattgatcggcacgatagatatgctaaccgcaacattgtgttctttgacatcaatatgaaaggtctagatggcattcaagggcctgtatatgttggcacgggatgtgtctttaacaggcaggcattatatggctatgatcctccagcatctgagaagcgagccaagatgacttgtgactgctggccaaagtggtgctgctgttgttgtggtggatcaaggaaaccaaagtctaagaaaaaaggacttaaggctttgctcgggttaggaggattgtatagcaagaagaagaaaatgatgggcaagcagtacactaggaagccatctggaccagtttttgaccttgaggaaattgaagaagggctggaaggatacgatgaattggaaaaatcatcactcatgtctcaaaagaattttgagaaacggtttgggatgtccccagttttcattacttccacacttatggaaaatggtggtcttcccgaggggactagcccgggatcactagtgaaagaagccattcatgttattagttgtggctatgaagaaaagactgaatggggcaaggagattggatggatttacggctcaattacagaagacatattgactggcttcaagatgcattgtagagggtggagatctgtatattgctgccccaagagacctgctttcaaaggatctgctccaataaatctttctgacaggttgcaccaagtcctgagatgggctcttggttctgttgaaatatttctgagtcgccattgtccgctttggtacggctatgggggaaagcttaaatatcttgaaagacttgcttatataaacaccattgtataccctttcacttcccttgctctgctagcctattgtacacttccagctgtttgtcttctgacaggaaaattcattgttccaactctgaacaaccttgcaagcatatggttcatggcccttttcctgtccatcatagccaccggtgtgcttgagctccgatggagtggagtaagcattgaggattggtggcgtaatgagcaattctgggtcatcggtggtctctcagctcatctatttgctgttttccaaggtcttcttaaggttcttgctggagtcgacacaaacttcacagttacatcaaaagcagctgatgatgctgagtttggggagctctacctcttcaaatggacaactcttctcattccaccaaccacaatcataatcctcaatatggtcggtgtggtggcaggagtttctgatgccataaacaatgggtatggctcatggggtcctctcttcggaaagctattctttgccttttgggttattgttcatctctatccattccttaagggtctcatggggaaacaaaacaggactcctacaattgtggttctatggtctattttgttggcctcaatcttctcacttgtgtgggttcgaatcgatcctttcttgcccaagcaaactggtccaattctcaagccatgtggtgtggagtgctaa

SEQ ID NO: 3 - protein, 1066 aa, Plantago ovata
MMESEGESKGKSLKSTGNQVCQICGDSVGLTVDGEPFAACDVCAFPVCRPCYEYERKDGNQSCPQCKTRYKRHKGSPAIRGDGEEDVSVEDGGSDLNHSNAHNDKQKLSERMLSWHMSYGRGDDNGAPKYDKEVSHNHIPLLTNGTDVSGELSAASPGRLSMASPPPSGGMKPRIVDPVREFGSPGLGNVAWKERVDGWKMKQDKPSVPMTSSHPPSERGLGDIDASTDIMGDDVMNDEARQPLSRKVSIPSSRINPYRMIIVLRLVILCIFLHYRITNPVPNAYSLWLTSVICEIWFAISWILDQFPKWLPINRETYLDRLSLRYEHEGEPSQLAAVDIFVSTVDPLKEPPLVTANTVLSILAVDYPVDKVSCYVSDDGAAMLSFESLSETAEFARKWVPFCKKYSIEPRAPEWYFTQKIDYLKDKVQPSFVKDRRAMKREYEEFKIRVNALVAKAQKIPEEGWTMQDGTPWPGNNTRDHPGMIQVFLGQSGGLDTDGNELPRLVYVSREKRPGYQHHKKAGAMNALVRVSAVLTNGPFLLNLDCDHYINNSKALREAMCFMMDPNLGKYVCYVQFPQRFDGIDRSDRYANRNTVFFDINLRGLDGIQGPVYVGTGCVFNRTALYGYEPPVKPKNKKPGMLSSCFGRSNKKSSRSNKKGSDKKKSGKHADPTVPVFSLEDIEEGVEGAGFDDEKSLLMSQMNLEKRFGGSAVFVASTLMENGGVPQSATPETLLKEAIHVISCGYEDKSEWGLEIGWIYGSVTEDILTGFKMHARGWRSIYCMPDRPAFKGSAPINLSDRLNQVLRWALGSVEILFSRHCPIWYGYSGRLKWLERFAYVNTTIYPITSIPLLCYCVLPAICLLTGKFIIPQISNLASIWFLSLFLSIFATGILEMRWSGVGIDEWWRNEQFWVIGGVSAHLFAVVQGLLKVLAGIDTNFTVTSKASDEGGDYAELYMFKWTTLLIPPTTLLILNLVGVVAGISYAINSGYQSWGPLFGKLFFSFWVIVHLYPFLKGLMGRQNRTPTVIVVWSILLASIFSLLWVRVDPFTSRVTGPAVELCGINC

SEQ ID NO: 4 - DNA, 3198 bp, Plantago ovata
atgatggaatctgaaggcgagagcaagggaaagagcctgaagagtacaggtaatcaggtctgtcagatctgtggggatagcgtgggactgacggtagatggtgagccatttgccgcctgcgatgtttgtgcatttccagtttgcaggccttgctatgaatatgagaggaaggacgggaatcagtcttgccctcagtgcaaaaccagatataaaagacataaaggaagtcctgcaattcgtggtgatggggaggaggatgtttctgttgaagatggaggtagtgatcttaatcactccaatgcacacaatgacaagcagaagctttcagagcgcatgttgagttggcacatgagttatgggcgaggggatgataatggggcaccaaagtatgataaggaggtctctcacaatcacatacctttgctcacaaatggaacagatgtttctggagagctctctgcagcatcccctgggcgcctttcaatggcatcgcctccaccttctggtggaatgaaacctagaattgtggatcctgtgagggagttcggatccccaggcttgggcaatgttgcctggaaagagagagttgatgggtggaagatgaagcaagataagccttcagttccaatgacctctagccatccaccctcagaaagaggacttggggatattgatgccagcactgatattatgggtgatgatgtaatgaatgatgaagctcggcagcctctatcgagaaaggtgtctattccatcatcgaggataaatccttacagaatgattattgtattgcggctcgtgattctgtgcattttcttgcattatcggataaccaatcccgtaccaaatgcatattccttgtggttgacttctgtaatctgtgagatctggtttgctatatcgtggattcttgatcagtttccaaaatggcttccaataaaccgtgaaacatatctcgacaggctttcactccgatatgaacatgaaggagagccatcccagttggctgctgttgacatatttgtcagtactgtcgatcctttgaaggagcctcctcttgttacagctaacactgtcctgtccattcttgcggtagactatccagtggacaaggtctcatgctatgtttctgatgatggtgctgccatgttgtcctttgaatctctctcagaaacagcagaatttgccagaaaatgggtccctttctgcaaaaagtacagcattgagccacgtgctccagaatggtattttacgcagaagatcgactacttgaaagataaagtccagccttcctttgtgaaagaccgtagggctatgaagagagaatatgaagagttcaaaatacgggtcaatgctctagttgccaaggcccaaaaaatccctgaagaaggctggaccatgcaagatggtactccatggcctggaaataatacaagagatcatccaggaatgattcaggtatttcttggtcaaagtggagggcttgacactgatggtaatgagctaccccggttagtctatgtctctcgtgaaaagcgaccaggataccaacatcacaagaaagccggtgctatgaatgcacttgttcgtgtgtcagcagttcttacaaatggacctttcttgttgaatcttgattgtgatcattacataaacaatagcaaggctttgcgtgaagcaatgtgcttcatgatggatccaaatcttggaaaatatgtgtgctatgttcagtttcctcaaagatttgatgggatagataggagtgatcgatatgccaacaggaacactgtcttttttgacattaacttgaggggtctagatggaattcaaggcccagtgtatgtgggcactggatgtgtcttcaacagaactgccttatatggttatgaacctcctgtgaagcctaaaaataagaagcctgggatgctctcttcttgttttggaaggtctaataaaaagagttccagatcaaataaaaaggggtcagataaaaagaaatctggcaaacatgctgatccaactgtacctgttttcagtttggaggatatagaggaaggtgttgaaggtgctggatttgatgatgaaaagtcactccttatgtcccaaatgaaccttgagaaaagatttggcgggtctgcggtatttgttgcatccaccttgatggagaatggaggtgtacctcagtctgctacacctgaaacccttctaaaagaggctattcatgttattagttgtgggtacgaagataagtcagaatggggccttgaaataggatggatttatggctctgttacagaagatattcttacaggatttaagatgcatgcacgtggttggcgttcaatttactgcatgcctgatagaccagcctttaagggatctgcaccaatcaatctttctgatcgtttgaaccaagtgcttcgatgggctttaggttccgttgagatcttgttcagcaggcattgtccgatatggtatggctatagtggaaggttgaaatggctcgagagatttgcttatgtcaacaccacaatataccctatcacatccattcctcttttatgttattgtgtgctaccagctatctgcttgcttactggaaagttcatcatccctcagattagtaaccttgccagtatttggtttctctccctctttctgtccatcttcgcaactggtatacttgaaatgagatggagtggagtaggtatagacgagtggtggagaaatgaacagttttgggtcattggaggtgtctcagctcatcttttcgctgtcgtccaaggtttgcttaaggtactggctggaattgataccaacttcactgtcacgtccaaagcttcagatgaaggtggagattatgcagaactttacatgttcaaatggactactctacttattccaccaactacacttctcatcttaaacttggttggtgttgttgctggaatttcatatgccataaacagcggttaccaatcctggggtccactttttgggaaattgttcttttccttctgggtgattgttcatctgtatcctttcttgaagggtctcatgggccgacagaacagaacccctactgttattgttgtgtggtcgattcttctggcctcgatcttctccttgttgtgggttagagtcgatcctttcacaagtagagtcacaggccctgctgtggagttgtgcggaatcaactgc

SEQ ID NO: 5 - protein, 1068 aa, Plantago ovata
MDSNAGLVAGSHNRNELVRIRHDSDSSVPKPLKNLNGQICQICGDTVGVTEGGDVFVACNECAFPVCRACYEYERKDGNQACPQCKTRYKRHKGSPRVDGDDDEDDVDDLENEFNSELSASSRRGTQQPIPLLTHGMPVSGEMPPSIQDTNSIRSTSGPLGPGDRVHSLPYVDPRRPVPVRIVDPTKDLNSYGLGNVDWKERVEGWKLKQDKSLGHVPNRYSEGKGDIEGTGSNGEELQMVDDLRQPMSRIIPISSSHLTPYRVIIVLRLIILGFFLQYRVTHPVKDAYPLWLTSVICEIWFALSWLLDQFPKWYPINRETYLDRLALRYDREGEPSQLVPIDVFVSTVDPLKEPPIVTANTVLSILSVDYPVDKVSCYVSDDGAAMLTFESLSETAEFARKWVPFCKKHNIEPRAPEFYFAKKIDYLKDKIQPSFVKERRAMKREYEEFKVRINALVAKAQKMPEEGWTMTDGTPWPGNKTRDHPGMIQVFLGHSGGLDTDGNELPRLVYVSREKRPGFQHHKKAGAMNALIRVSAVLTNGAYILNVDCDHYFNNSKALKEAMCFMMDPAYGKKTCYVQFPQRFDGIDLHDRYANRNIVFFDINLKGLDGLQGPVYVGTGCCFNRQALYGYDPVLTEEDLQPNIVVKSCCGTRKKKTANKKYIDKKRAAKRTESTLPIFNMEDVEEGVEGYEEEKSLLMSQKNLEKRFGQSPVFIAATFMEQGGIPASTNTSTLLREAIHVLSCGYEDKTEWGKEIGWIYGSVTEDILTGFKMHTRGWISIYCMPPRPAFKGSAPINLSDRLNQVLRWALGSIEILLSRHCPIWYGYEGGRLQVLERLAYINTIVYPLTSIPLIAYCILPAVCLLTNKFIIPQISNFASMWFILLFVSIFATGILEMRWGGVSVEDWWRNEQFWVIGGTSAHLFAVFQGLLKVLAGIDTNFTVTSKAADEEGDFAELYIFKWTSLLIPPTTVLIINMVGIVAGVSAAINSGYQSWGPLFGKLFFAIWVIVHLYPFLKGLLGRQNRTPTIVIVWSILLASIFSLLWVRIDPFTSTDTTSTVGQCGIDC

SEQ ID NO: 6 - DNA, 3204 bp, Plantago ovata
atggattctaatgccggattggtagctggatcgcataacaggaacgagcttgttagaatccgccatgactctgatagtagtgtgccgaagcccctgaagaacttgaatgggcagatatgtcagatttgtggagatactgtcggagtgactgaaggtggggacgttttcgttgcttgcaatgagtgtgcatttccagtatgcagagcttgttatgagtatgagcgaaaagatggaaatcaggcatgcccccagtgcaagactaggtacaagaggcacaaagggagtccgagggttgatggtgatgatgatgaggatgacgttgatgatctggagaatgaattcaattccgagttgtctgcgtcttctaggcgtggaactcagcaaccaatccctcttcttacccatgggatgccagtctctggtgaaatgccgccttctattcaagatactaactccattagaagtacatctggacctttaggccctggagaccgtgttcattcccttccttacgtcgatccacgtcggccagttcctgtgagaattgttgatcctaccaaggacttgaactcttatggacttgggaatgttgactggaaggaaagagtggaaggttggaagttaaagcaggataaaagtctggggcatgttccgaatagatacagtgaagggaaaggagacattgaaggcactgggtcaaatggtgaagaactacaaatggttgatgatttacgacaacctatgagccgaatcatccccatttcctcctcccatcttactccttatcgtgttatcattgttcttcggttgatcattctggggtttttcttacaataccgtgtcactcacccagtgaaggatgcttatcccttgtggttgacatctgttatctgtgagatttggtttgccttatcttggctcttggatcagttcccaaaatggtaccctatcaaccgtgagacatatcttgacagacttgcattgaggtatgacagagaaggcgagccttctcagttagtaccaatcgatgtctttgtgagtacagtggatccgcttaaagagcctcctattgttactgccaacactgtgctgtccatcctttcagtggattacccggtcgacaaagtctcatgttatgtttcagatgatggtgctgcgatgcttacatttgagtccctatctgaaactgcagagtttgcacggaaatgggtgccgttctgtaagaaacataatattgagcctagagcccctgaattctattttgctaagaagatagattacctgaaggacaagattcagccgtcctttgtgaaagaacgcagagccatgaagagagaatatgaagaattcaaggttcgaattaatgctctcgtagcaaaagctcaaaagatgcctgaagaaggctggacaatgacagatggaactccatggccaggaaataaaacaagggatcatccaggaatgatccaggtatttcttggacatagtggtggccttgatacagatggtaacgagctacctcgtttggtctatgtttcgcgtgagaagcgtccaggtttccaacatcacaagaaagctggagccatgaatgctctgattcgtgtctctgctgtcctaacaaatggagcatacattctaaatgtcgattgtgatcactacttcaacaacagcaaagctcttaaagaagccatgtgctttatgatggaccctgcttatggaaagaagacttgctatgtgcagttcccacaacgtttcgatggcattgatttgcacgatcgatatgccaatcgcaatattgtcttttttgatattaacttgaaaggactggatggccttcagggtcctgtttatgtgggaactggatgttgctttaacaggcaggcactgtatgggtatgacccagttctaacagaagaagacttgcaaccaaacatcgttgttaaaagctgctgcggcacaaggaagaaaaagactgccaataagaaatacattgataagaagcgagcagccaaaagaaccgagtcaacccttccaatatttaacatggaggatgttgaagagggtgttgaaggatatgaagaggagaagtctcttcttatgtctcagaagaacttggagaaacggtttgggcaatctcctgtcttcatcgctgcaacatttatggaacagggaggcattcctgcatcaacaaatactagtactcttttgagagaagcgatccatgtccttagctgcgggtacgaagataaaacggagtggggcaaagagattgggtggatctacggctctgtgacagaagatattttaacagggtttaagatgcatactcgagggtggatttccatttactgtatgcctcctcgaccagctttcaagggttctgctccaatcaatctttcggatcgtctgaatcaggtccttcgatgggctctggggtccattgaaattctgctgagcaggcattgtcctatatggtatggctacgaaggtggcaggctacaggtcttggagaggctggcgtatatcaacaccatcgtgtatccgctcacatccatccctctcattgcttactgtattcttcctgccgtctgtcttctcacaaacaagttcattattccccagataagcaatttcgctagcatgtggtttattctcctcttcgtctccattttcgctactggtattcttgaaatgaggtggggtggtgttagtgttgaggactggtggagaaacgagcagttctgggtcatcggaggaacatctgctcatctcttcgctgtcttccaaggtctcctcaaagtacttgctggaattgacaccaacttcactgtcacatccaaggcagcagatgaagagggagatttcgcggagctgtacattttcaagtggacttccctccttattcctccaacgactgtcctcatcataaacatggtcggaattgtagccggtgtttctgctgccatcaacagtgggtaccaatcatggggtccacttttcggtaagctgttctttgccatttgggtgattgtccatctctacccattcttgaagggtttgttgggcagacagaaccgtacccctaccattgtcatcgtctggtccattctccttgcatcgatattctccctgttgtgggtgcgtatcgacccatttacatctactgatacaacatctacggtagggcaatgtggaatcgactgc